(19) United States
(12) Patent Application Publication
(10) Pub. No.: US 2019/0125499 A1
(43) Pub. Date: May 2, 2019

(54) BONE GRAFT INJECTION DEVICE

(71) Applicant: MAXILLENT LTD., Herzliya (IL)

(72) Inventors: Ilan UCHITEL, Kefar Saba (IL); Adi ALPHANDARY, Hod Hasharon (IL); Gideon FOSTICK, Givat Shmuel (IL); Yossi GROSS, Moshav Mazor (IL); Zev SOHN, Ginot Shomron (IL); Leon GINZBURG, Kefar Saba (IL)

(73) Assignee: MAXILLENT LTD., Herzliya (IL)

(21) Appl. No.: 16/094,745

(22) PCT Filed: Apr. 20, 2017
(86) PCT No.: PCT/IL2017/050463

§ 371 Date: Oct. 18, 2018

Related U.S. Application Data
(63) Continuation-in-part of application No. PCT/IL2016/050423, filed on Apr. 20, 2016.
(60) Provisional application No. 62/412,985, filed on Oct. 26, 2016.

(30) Foreign Application Priority Data
Apr. 20, 2016 (IL) PCT/IL2016/050423

(51) Int. Cl. A61C 8/00; A61C 1/00; A61C 1/08

(52) U.S. Cl. CPC A61C 8/0092; A61C 1/0015; A61F 2/4601; A61C 1/087; A61C 1/0092

(57) ABSTRACT

A composition delivery source (200) is provided that includes (a) a chamber (202), which is shaped so as to define (A) one or more liquid ports (310) in fluid communication with an interior of the chamber (202), and (B) one or more solid-liquid composition ports (312) in fluid communication with the interior of the chamber (202); (b) a solid-liquid composition delivery tube (314) in fluid communication with at least one of the one or more solid-liquid composition ports (312); (c) a mixing tube (316) in fluid communication with at least one of the one or more liquid ports (310) and at least one of the one or more solid-liquid composition ports (312); and (d) a liquid-supply tube (318) in fluid communication with at least one of the one or more liquid ports (310). A pump unit (201) includes one or more pumps (223), which are arranged to cause flow in the mixing tube (316) during a mixing activation state (342), and to cause flow in the liquid-supply tube (318) during a particle-delivery activation state (344).

### CROSS-REFERENCE TO RELATED APPLICATIONS

The present application (a) claims priority from U.S. Provisional Application 62/412,985, filed Oct. 26, 2016, and (b) claims priority and is a continuation-in-part of International Application PCT/IL2016/050423, filed Apr. 20, 2016. Both of the above-referenced applications are assigned to the assignee of the present application and are incorporated herein by reference.

### FIELD OF THE APPLICATION

The present invention relates generally to surgical tools and implantation methods, and specifically to minimally-invasive surgical tools and implantation methods.

### BACKGROUND OF THE APPLICATION

Osseointegrated dental implants are typically metallic or ceramic screws that are placed in the jawbone for supporting artificial teeth after the loss of natural teeth. Replacement of the maxillary teeth is often a challenging surgical procedure when the remaining maxillary bone has insufficient height to support the implant. One surgical technique for augmenting the maxillary bone includes injecting a regenerative material, such as autogenic, allogeneic, xenogeneic, or synthetic bone grail, into the vicinity of the maxillary bone. The regenerative material forms additional bone mass that integrates with the existing maxillary bone, providing the necessary alveolar height to support the implant.

Bone augmentation procedures are often surgically difficult to perform, and are associated with complications, including infection of the maxillary sinus. The top of the maxillary alveolar ridge forms the floor of the maxillary sinus, and is covered by a thin membrane known as the Schneiderian or subantral membrane. In one surgical procedure, known as a closed or internal sinus lift or elevation procedure, the surgeon drills a bore through the maxillary alveolar ridge from the oral cavity at the desired location of the implant. The bore penetrates the ridge to below the Schneiderian membrane. The surgeon injects the regenerative material through the bore to below the membrane, forming a cavity defined by the top of the ridge and the bottom of the membrane, which cavity occupies a portion of the space initially occupied by the maxillary sinus.

To prevent potentially serious complications, the surgeon must be careful not to perforate the Schneiderian membrane. This is often difficult, because of the delicacy of the membrane, and the restricted access afforded by the closed approach.

Hydraulic sinus lifting is performed by applying hydraulic pressure between the sinus floor and the Schneiderian membrane. The hydraulic elevation can be performed via a crestal or lateral approach. Once the membrane is elevated, using a hydraulic, closed, or lateral window technique, a bone graft material is applied, typically using one of two conventional techniques. The first conventional technique is the mechanical insertion of bone graft, which is formulated in small particles. This technique is manually demanding, and it may cause application of unequal stresses to the membrane, which may result in perforation of the membrane. The second conventional technique is the injection of bone graft in a gel formulation by applying the same principles of hydraulic elevation used during raising of the membrane.

### SUMMARY

Some embodiments of the present invention provide surgical tools and methods for use in conjunction with minimally-invasive sinus lift techniques for performing a bone augmentation procedure on the maxillary alveolar ridge while reducing the risk of perforating the Schneiderian membrane and of infection.

For some applications, a bone graft injection system is provided that comprises a composition delivery source, which comprises a chamber, a solid-liquid composition delivery tube, a mixing tube, and a liquid-supply tube. The chamber is shaped so as to define (a) one or more liquid ports in fluid communication with an interior of the chamber, and (b) one or more solid-liquid composition ports in fluid communication with the interior of the chamber. The solid-liquid composition delivery tube is in fluid communication with at least one of the one or more solid-liquid composition ports. The mixing tube is in fluid communication with at least one of the one or more liquid ports and at least one of the one or more solid-liquid composition ports. The liquid-supply tube is in fluid communication with at least one of the one or more liquid ports.

For some applications, the surgical tool further comprises a pump unit, which comprises one or more pumps, which are arranged (a) to cause flow in the mixing tube during a mixing activation state, and (b) to cause flow in the liquid-supply tube during a particle-delivery activation state. Typically, the pump unit is arranged to cause, in the mixing tube, flow that raises the solid bone graft particles in a puff into the physiological liquid solution in the chamber.

For some applications, the one or more pumps comprise (a) a mixing pump, which is arranged to cause the flow in the mixing tube during the mixing activation state, and (b) a liquid-supply pump, which is arranged to cause the flow in the liquid-supply tube during the particle-delivery activation state. For some applications, the pump unit further comprises control circuitry, which is configured to repeatedly:
assume the mixing activation state, in which the control circuitry activates the mixing pump to mix the solid particles and the physiological liquid solution in the chamber to form a solid-liquid composition, by pumping the physiological liquid solution through the mixing tube and into the chamber, and
assume the particle-delivery activation state, wherein the control circuitry, during at least a portion of the particle-delivery activation state, activates the liquid-supply pump to apply positive pressure to pump the solid-liquid composition from the chamber into the solid-liquid composition delivery tube.

For some applications, the one or more pumps comprise a pump (e.g., exactly one pump) that is arranged to (a) cause the flow in the mixing tube during the mixing activation state, and (b) cause the flow in the liquid-supply tube during the particle-delivery activation state. For some of these applications, the pump unit further comprises one or more valves, which are arranged to regulate flow in the liquid-supply tube and in the mixing tube. For some applications, the one or more valves comprise (a) a liquid-supply-tube valve, which is arranged to regulate flow in the liquid-supply tube, and (b) a mixing-tube valve, which is arranged to regulate flow in the mixing tube. For some applications, the pump unit further comprises control circuitry, which is configured to repeatedly:
assume the mixing activation state, in which the control circuitry activates the pump to mix the solid particles and the physiological liquid solution in the chamber to form a solid-liquid composition, by pumping the physiological liquid solution through the mixing tube and into the chamber, and
assume the particle-delivery activation state, wherein the control circuitry, during at least a portion of the particle-delivery activation state, activates the pump to apply positive pressure to pump the solid-liquid composition from the chamber into the solid-liquid composition delivery tube.

For some applications, the control circuitry is configured to (a) during the mixing activation state, close the liquid-supply-tube valve and open the mixing-tube valve, and (b) during the particle-delivery activation state, open the liquid-supply-tube valve and close the mixing-tube valve.

For some applications, the control circuitry is configured, during each of one or more negative-positive particle delivery cycles of the particle-delivery activation state, to assume:
a negative particle-delivery activation sub-state, in which the control circuitry activates one of the one or more pumps to apply negative pressure to pump liquid from the solid-liquid composition delivery tube toward the chamber, and
a positive particle-delivery activation sub-state, in which the control circuitry activates the one of the one or more pumps to apply the positive pressure to pump the solid-liquid composition from the chamber into the solid-liquid composition delivery tube, wherein a direction of pumping of the one of the one or more pumps in the positive particle-delivery activation sub-state is opposite a direction of pumping of the one of the one or more pumps in the negative particle-delivery activation sub-state.

For some applications, the surgical tool comprises an automated device that both prepares (e.g., mixes) and delivers the solid-liquid composition during the procedure.

There is therefore provided, in accordance with an application of the present invention, apparatus for use with solid particles and a liquid container containing a physiological liquid solution, the apparatus including:

(1) a composition delivery source, which includes:
(a) a chamber, which is shaped so as to define (A) one or more liquid ports in fluid communication with an interior of the chamber, and (B) one or more solid-liquid composition ports in fluid communication with the interior of the chamber;
(b) a solid-liquid composition delivery tube, which is in fluid communication with at least one of the one or more solid-liquid composition ports;
(c) a mixing tube, which is in fluid communication with at least one of the one or more liquid ports and at least one of the one or more solid-liquid composition ports; and
(d) a liquid-supply tube, which is in fluid communication with at least one of the one or more liquid ports; and

(2) a pump unit, which includes one or more pumps, which are arranged:
(a) to cause flow in the mixing tube during a mixing activation state, and
(b) to cause flow in the liquid-supply tube during a particle-delivery activation state.

For some applications, the solid particles are solid bone graft particles, and the apparatus is for use with the solid bone graft particles.

For some applications, the pump unit is arranged to cause, in the mixing tube, flow that raises the solid particles in a puff into the physiological liquid solution in the chamber.

For some applications, the mixing tube (a) merges with the liquid-supply tube at an exit junction, and (b) is in fluid communication with the at least one of the one or more liquid ports via a portion of the liquid-supply tube.

For some applications, the liquid-supply tube (a) merges with the mixing tube at an exit junction, and (b) is in fluid communication with the at least one of the one or more liquid ports via a portion of the mixing tube.

For some applications, the mixing tube (a) merges with the solid-liquid composition delivery tube at a return junction, and (b) is in fluid communication with the at least one of the one or more solid-liquid composition polls via a portion of the solid-liquid composition delivery tube.

For some applications:

the one or more solid-liquid composition ports include (a) a solid-liquid composition delivery port and (b) a solid-liquid composition inlet port,

the mixing tube is in fluid communication with the solid-liquid composition inlet port, and

the solid-liquid composition delivery tube is in fluid communication with the solid-liquid composition delivery port.

For some applications, a length of the solid-liquid composition delivery tube equals at least 500% of a length of the mixing tube. For some applications, a length of the solid-liquid composition delivery tube equals at least 300% of a sum of a length of the mixing tube and a length of the liquid-supply tube. For some applications, a length of the solid-liquid composition delivery tube is at least 50 cm. For some applications, a length of the solid-liquid composition delivery tube is at least 100 cm.

For some applications, the chamber is shaped so as to define exactly one liquid port, and exactly one solid-liquid composition port.

For some applications, the one or more pumps are one or more peristaltic pumps, respectively.

For some applications, the one or more pumps include:

a mixing pump, which is arranged to cause the flow in the mixing tube during the mixing activation state; and

a liquid-supply pump, which is arranged to cause the flow in the liquid-supply tube during the particle-delivery activation state.

For some applications, the pump unit further includes control circuitry, which is configured to repeatedly:

(a) assume the mixing activation state, in which the control circuitry activates the mixing pump to mix the solid particles and the physiological liquid solution in the chamber to form a solid-liquid composition, by pumping the physiological liquid solution through the mixing tube arid into the chamber, and

(b) assume the particle-delivery activation slate, wherein the control circuitry, during at least a portion of the particle-delivery activation state, activates the liquid-supply pump to apply positive pressure to pump the solid-liquid composition from the chamber into the solid-liquid composition delivery tube.

For some applications, the liquid-supply pump is a liquid-supply peristaltic pump, which includes a rotor.

For some applications, the control circuitry is configured to assume the particle-delivery activation state a plurality of times in alternation with mixing activation states, and to begin each of the particle-delivery activation states with the rotor at a same rotational position.

For some applications, the mixing pump is a mixing peristaltic pump, which includes a rotor.

For some applications:

the mixing peristaltic pump includes a total number of rollers equal to at least two, and

the control circuitry is configured to assume the mixing activation state a plurality of times in alternation with particle-delivery activation states, and to begin the mixing activation states with the rotor at respective starting rotational positions, which are identical to one another or rotationally offset from one another by the product of (a) 360 degrees divided by the total number of rollers and (b) a positive integer.

For some applications:

the mixing peristaltic pump includes (a) a pump casing that is shaped so as to define a partial-circle mixing tube channel in which the mixing tube is disposed, and (b) an odd total number of rollers, the odd total number equal to at least one, and

the control circuitry is configured to assume the mixing activation state a plurality of times in alternation with particle-delivery activation states, and to begin each of the mixing activation states with an aligned total number of the rollers rotationally aligned with the mixing tube channel, the aligned total number equal to more than half of the odd total number.

For some applications, the odd total number equals at least three.

For some applications, the mixing pump and the liquid-supply pump are respective peristaltic pumps.

For some applications, the pump unit includes exactly one pump, which is arranged to:

(a) cause the flow in the mixing tube during the mixing activation state, and

(b) cause the flow in the liquid-supply tube during the particle-delivery activation state.

For some applications, the one or more pumps include a pump that is arranged to:

(a) cause the flow in the mixing tube during the mixing activation state, and

(b) cause the flow in the liquid-supply tube during the particle-delivery activation state.

For some applications, the pump unit further includes one or more valves, which are arranged to regulate flow in the liquid-supply tube and in the mixing tube.

For some applications, the one or more valves include:

a liquid-supply-tube valve, which is arranged to regulate flow in the liquid-supply tube; and

a mixing-tube valve, which is arranged to regulate flow in the mixing tube.

For some applications, the pump unit further includes control circuitry, which is configured to repeatedly:

(a) assume the mixing activation state, in which the control circuitry activates the pump to mix the solid particles and the physiological liquid solution in the chamber to form a solid-liquid composition, by pumping the physiological liquid solution through the mixing tube and into the chamber, and

(b) assume the particle-delivery activation state, wherein the control circuitry, during at least a portion of the particle-delivery activation state, activates the pump to apply positive pressure to pump the solid-liquid composition from the chamber into the solid-liquid composition delivery tube.

For some applications:

the pump unit further includes (a) a liquid-supply-tube valve, which is arranged to regulate flow in the liquid-supply tube, and (b) a mixing-tube valve, which is arranged to regulate flow in the mixing tube, and

the control circuitry is configured to:
(a) during the mixing activation state, close the liquid-supply-tube valve and open the nixing-tube valve, and
(b) during the particle-delivery activation state, open the liquid-supply-tube valve and close the mixing-tube valve.

For some applications, the pump is a peristaltic pump, which includes a rotor.

For some applications, the pump unit further includes control circuitry, which is configured to repeatedly:

(a) assume a mixing activation state, in which the control circuitry activates one of the one or more pumps to mix the solid particles and the physiological liquid solution in the chamber to form a solid-liquid composition, by pumping the physiological liquid solution through the mixing tube and into the chamber, and

(b) assume a particle-delivery activation state, wherein the control circuitry, during at least a portion of the particle-delivery activation state, activates one of the one or more pumps to apply positive pressure to pump the solid-liquid composition from the chamber into the solid-liquid composition delivery tube.

For some applications, the control circuitry is configured to assume the mixing activation state and the particle-delivery activation state at the same time.

For some applications, the control circuitry is configured to assume the mixing activation state and the particle-delivery activation state at partially-overlapping times.

For some applications, the control circuitry is configured to assume the mixing activation state and the particle-delivery activation state at non-overlapping times.

For some applications, the control circuitry is configured, during each of one or more negative-positive particle delivery cycles of the particle-delivery activation state, to assume:

a negative particle-delivery activation sub-state, in which the control circuitry activates one of the one or more pumps to apply negative pressure to pump liquid from the solid-liquid composition delivery tube toward the chamber, and

a positive particle-delivery activation sub-state, in which the control circuitry activates the one of the one or more pumps to apply the positive pressure to pump the solid-liquid composition from the chamber into the solid-liquid composition delivery tube, wherein a direction of pumping of the one of the one or more pumps in the positive particle-delivery activation sub-state is opposite a direction of pumping of the one of the one or more pumps in the negative particle-delivery activation sub-state.

For some applications, the control circuitry is configured to assume the negative particle-delivery activation sub-state for a first duration during each of the one or more negative-positive particle delivery cycles, and to assume the positive particle-delivery activation sub-state for a second duration during each of the one or more negative-positive particle delivery cycles, the second duration greater than the first duration.

For some applications, the control circuitry is configured to assume the mixing activation state and the particle-delivery activation state at non-overlapping times.

For some applications, the control circuitry is configured to assume the mixing activation state and the negative particle-delivery activation sub-state at partially-overlapping times.

For some applications, the control circuitry is configured to assume the particle-delivery activation state in a plurality of particle-delivery-state cycles, and to begin the particle-delivery activation state in each of the particle-delivery-state cycles with the negative particle-delivery activation sub-state.

For some applications, the control circuitry is configured to assume the particle-delivery activation state in a plurality of particle-delivery-state cycles, and to begin the particle-delivery activation state in each of the particle-delivery-state cycles with the positive particle-delivery activation sub-state.

For some applications, the control circuitry is configured to provide a plurality of the negative-positive particle delivery cycles during the particle-delivery activation state.

For some applications:

the one of the one or more pumps is a peristaltic pump, which includes a rotor,

the peristaltic pump is capable of (a) pumping fluid at an average rate throughout a full 360-degree revolution of the rotor at a certain speed, and (b) pumping fluid at a maximum rate during portions of the full 360-degree revolution at the certain speed, the maximum rate greater than the average rate, and

the control circuitry is configured, when in both the positive and the negative particle-delivery activation sub-states, to activate the peristaltic pump to (a) rotate the rotor, at the certain speed, a partial revolution equal to a fraction of the full 360-degree revolution of the rotor, the fraction less than 1, and (b) pump, throughout the partial revolution, the fluid at the maximum rate.

For some applications:

the one of the one or more pumps is a peristaltic pump, which includes a rotor, and

the control circuitry is configured:
when in the positive particle-delivery activation sub-state, to activate the peristaltic pump to rotate the rotor, in a first rotational direction, a first partial revolution equal to a fraction of a full 360-degree revolution of the rotor, the fraction less than 1, and
when in the negative particle-delivery activation sub-state, to activate the peristaltic pump to rotate the rotor, in a second rotational direction opposite the first rotational direction, a second partial revolution equal to the fraction of the full 360-degree revolution of the rotor.

For some applications:

the one of the one or more pumps is a peristaltic: pump, which includes a rotor, and

the control circuitry is configured, when in the positive particle-delivery activation sub-state, to activate the peristaltic pump to:
rotate the rotor a partial revolution equal to a fraction of a full 360-degree revolution of the rotor, the fraction less than 1, and
pump, throughout the partial revolution, a volume of fluid that is greater than the product of the fraction and a volume of fluid pumpable throughout the full 360-degree revolution of the rotor.

For some applications, the apparatus further includes a shaft unit, which includes a. shaft delivery tube in fluid communication with a distal end of the solid-liquid composition delivery tube.

For some applications, the shaft unit further includes a removable depth limiting element, which is configured to limit a depth of insertion of the shaft delivery tube into a bore through a bone when the shaft delivery tube is inserted into the bore.

For some applications:

the shaft unit includes a shaft delivery tube,

the shaft unit further includes a sealing element disposed around an external surface of the shaft delivery tube, and

the depth limiting element is removable from the shaft unit without removal of the sealing element.

There is further provided, in accordance with an application of the present invention, apparatus for use with a liquid container containing a physiological liquid solution, the apparatus including a composition delivery source, which includes:

(a) a chamber, which:
(i) includes solid bone graft particles, and
(ii) is shaped so as to define (A) one or more liquid ports in fluid communication with an interior of the chamber, and (B) one or more solid-liquid. composition ports in fluid communication with the interior of the chamber;

(b) a solid-liquid composition delivery tube, which is in fluid communication with at least one of the one or more solid-liquid composition ports;

(c) a mixing tube, which is in fluid communication with at least one of the one or more liquid ports and at least one of the one or more solid-liquid composition ports; and

(d) a liquid-supply tube, which is in fluid communication with at least one of the one or more liquid ports.

There is still further provided, in accordance with an application of the present invention, apparatus for use with solid particles and a liquid container containing a physiological liquid solution, the apparatus including a pump unit, which includes:

(a) one or more pumps; and

(b) control circuitry, which is configured to repeatedly:
(i) assume a mixing activation state, in which the control circuitry activates one the one or more pumps, and
(ii) assume a particle-delivery activation state,

wherein the control circuitry is configured, during each of one or more negative-positive particle delivery cycles of the particle-delivery activation state, to assume:
a negative particle-delivery activation sub-state, in which the control circuitry activates one of the one or more pumps apply negative pressure to pump in a first direction, and
thereafter, a positive particle-delivery activation sub-state, in which the control circuitry activates the one of the one or more pumps to apply positive pressure to pump in a second direction opposite the first direction.

For some applications:

the one or more pumps include (a) a mixing pump and (b) a liquid-supply pump,

the control circuitry is configured to activate the mixing pump during the mixing activation state, and

the control circuitry is configured, during each of the one or more negative-positive particle delivery cycles of the particle-delivery activation state, to assume:
the negative particle-delivery activation sub-state, in which the control circuitry activates the liquid-supply pump apply negative pressure to pump in the first direction, and
thereafter, the positive particle-delivery activation sub-state, in which the control circuitry activates the liquid-supply pump to apply positive pressure to pump in the second direction opposite the first direction.

For some applications:

the pump unit includes exactly one pump,

the control circuitry is configured to activate the exactly one pump during the mixing activation state, and

the control circuitry is configured, during each of the one or more negative-positive particle delivery cycles of the particle-delivery activation state, to assume:
the negative particle-delivery activation sub-state, in which the control circuitry activates the exactly one pump to apply negative pressure to pump in the first direction, and
thereafter, the positive particle-delivery activation sub-state, in which the control circuitry activates the exactly one pump to apply positive pressure to pump in the second direction opposite the first direction.

For some applications:

the control circuitry is configured to activate one of the one or more pumps during the mixing activation state, and

the control circuitry is configured, during each of the one or more negative-positive particle delivery cycles of the particle-delivery activation state, to assume:
the negative particle-delivery activation sub-state, in which the control circuitry activates the one of the one or more pumps to apply negative pressure to pump in the first direction, and
thereafter, the positive particle-delivery activation sub-state, in which the control circuitry activates the one of the one or more pumps to apply positive pressure to pump in the second direction opposite the first direction.

For some applications:

the pump unit further includes (a) a liquid-supply-tube valve, which is arranged to regulate flow in the liquid-supply tube, and (b) a mixing-tube valve, which is arranged to regulate flow in the mixing tube, and

the control circuitry is configured to:
(a) during the mixing activation state, close the liquid-supply-tube valve and open the mixing-tube valve, and
(b) during the particle-delivery activation state, open the liquid-supply-tube valve and close the mixing-tube valve.

There is additionally provided, in accordance with an application of the present invention, apparatus for use with solid particles and a physiological liquid solution, the apparatus including:

a composition delivery source, which includes:
(a) a chamber, which is shaped so as to define one or more liquid ports and one or more solid-liquid composition ports;
(b) a solid-liquid composition delivery tube, which is in fluid communication with at least one of the one or more solid-liquid composition ports; and
(c) a mixing tube, which is in fluid communication with at least one of the one or more liquid ports and at least one of the one or more solid-liquid composition ports; and

a pump unit, which includes one or more pumps, and which is arranged to cause, in the mixing tube, flow that raises the solid particles in a puff into the physiological liquid solution in the chamber.

For some applications, the solid particles are solid bone graft particles, and the apparatus is for use with the solid bone graft particles.

For some applications, a length of the solid-liquid composition delivery tube equals at least 500% of a length of the mixing tube and/or at least 50 cm.

For some applications, the apparatus further includes the solid particles.

For some applications, the apparatus is for use with a liquid container, and the composition delivery source further includes a liquid-supply tube, which is in fluid communication with at least one of the one or more liquid ports, and is coupled in fluid communication with an interior of the liquid solution container.

For some applications, the one or more pumps are arranged to:

(a) cause, in the mixing tube, the flow that raises the solid particles in the puff into the physiological liquid solution in the chamber, during a mixing activation state, and

(b) cause flow in the liquid-supply tube, during a particular-delivery activation state.

For some applications, the pump unit includes exactly one pump, which is arranged to:

(a) cause, in the mixing tube, the flow that raises the solid particles in the puff into the physiological liquid solution in the chamber, during the mixing activation state, and

(b) cause flow in the liquid-supply tube, during the particular-delivery activation state.

For some applications, the one or more pumps include a pump that is arranged to:

(a) cause, in the mixing tube, the flow that raises the solid particles in the puff into the physiological liquid solution in the chamber, during the mixing activation state, and

(b) cause flow in the liquid-supply tube, during the particular-delivery activation state.

For some applications, the pump unit further includes one or more valves, which are arranged to regulate flow in the liquid-supply tube and in the mixing tube.

For some applications, the one or more valves include:

a liquid-supply-tube valve, which is arranged to regulate flow in the liquid-supply tube; and

a mixing-tube valve, which is arranged to regulate flow in the mixing tube.

For some applications, the one or more pumps include:

a mixing pump, which is arranged to cause, in the mixing tube, the flow that raises the solid particles in the puff into the physiological liquid solution in the chamber, during the mixing activation state; and

a liquid-supply pump, which is arranged to cause flow in the liquid-supply tube, during the particular-delivery activation state.

There is yet additionally provided, in accordance with an application of the present invention, a method for use with solid particles and a liquid container containing a physiological liquid solution, the method including:

providing a composition delivery source, which includes (a) a chamber, which is shaped so as to define (A) one or more liquid ports in fluid communication with an interior of the chamber, and (B) one or more solid-liquid composition ports in fluid communication with the interior of the chamber; (b) a solid-liquid composition delivery tube, which is in fluid communication with at least one of the one or more solid-liquid composition ports; (c) a mixing tube, which is in fluid communication with at least one of the one or more liquid ports and at least one of the one or more solid-liquid composition ports; and (d) a liquid-supply tube, which is in fluid communication with at least one of the one or more liquid ports;

providing a pump unit, which includes one or more pumps, which are arranged (a) to cause flow in the mixing tube during a mixing activation state, and (b) to cause flow in the liquid-supply tube during a particle-delivery activation state;

inserting, from a First side of a maxillary bone of a jaw, a shaft delivery tube of a shaft unit into a bore that passes through the maxillary bone from the first side to a second side of the maxillary bone, such that a distal opening of the shaft delivery tube is disposed in the bore or in a cavity that is (a) adjacent to the second side of the maxillary bone and (b) between the second side of the maxillary bone and a Schneiderian membrane, wherein the distal opening is in fluid communication with the delivery tube, and the shaft delivery tube is in fluid communication with a distal end of the solid-liquid composition delivery tube; and

activating the pump unit to:
provide a solid-liquid composition of (a) the solid particles and (b) the physiological liquid solution, and
inject the solid-liquid composition through the distal opening via the shaft delivery tube and the solid-liquid composition delivery tube.

For some applications, the solid particles are solid bone graft particles, and activating the pump unit to provide the solid-liquid composition includes activating the. pump unit to provide the solid-liquid composition of (a) the solid bone graft particles and (b) the physiological liquid solution.

For some applications, the method further includes raising the Schneiderian membrane to form the cavity.

For some applications:

inserting the shaft delivery tube includes positioning the distal opening at a solid-liquid-composition-delivery location,

raising the Schneiderian membrane includes:
positioning the distal opening at a liquid-delivery location that is within the bore or within 1 mm above the bore: and
while the distal opening is positioned at the liquid-delivery location, injecting the physiological liquid solution to raise the Schneiderian membrane, and

positioning the distal opening at the solid-liquid-composition-delivery location includes positioning the distal opening at the solid-liquid-composition-delivery location after finishing injecting the physiological liquid solution to raise the Schneiderian membrane.

There is also provided, in accordance with an application of the present invention, a method for use with solid particles and a physiological liquid solution, the method including:

providing a composition delivery source, which includes (a) a chamber, which is shaped so as to define one or more liquid ports and one or more solid-liquid composition ports; (b) a solid-liquid composition delivery tube, which is in fluid communication with at least one of the one or more solid-liquid composition ports; and (c) a mixing tube, which is in fluid communication with at least one of the one or more liquid ports and at least one of the one or more solid-liquid composition ports; and

activating a pump unit, which includes one or more pumps, to cause, in the mixing tube, flow that raises the solid particles in a puff into the physiological liquid solution in the chamber, thereby forming a solid-liquid composition.

There is further provided, in accordance with an application of the present invention, a method including:

injecting, from a first side f a bone, through (a) a bore that passes through the bone from the first side to a second side of the bone, and (b) into a cavity adjacent to tile second side of the bone, a solid-liquid composition of solid particles and a physiological liquid solution; and

draining, from the cavity and through the bore, the physiological liquid solution of the solid-liquid composition, while inhibiting passage of the solid particles of the solid-liquid composition, such that the solid particles accumulate in the cavity.

There is still further provided, in accordance with an application of the present invention, apparatus comprising:

a fluid-delivery assembly removably coupleable to an osteotome, the fluid-delivery assembly comprising a locking element, configured to lock the osteotome to the fluid-delivery assembly in a locked state and to release the osteotome from the fluid-delivery assembly in an unlocked state,

wherein a channel of the osteotome is in sealed fluid communication with the fluid-delivery assembly when in the locked state, and

wherein the fluid-delivery assembly can rotate about a central longitudinal axis of the osteotome while in the locked state.

For some applications, the locking element allows fast coupling and decoupling of the osteotome to and from the fluid-delivery assembly.

The present invention will be more fully understood from the following detailed description of embodiments thereof, taken together with the drawings, in which:

### BRIEF DESCRIPTION OF THE DRAWINGS

FIGS. 1A-E are schematic illustrations of bone graft injection systems for the insertion of solid bone graft particles into a cavity, in accordance with respective applications of the present invention;

FIGS. 2A-C are diagrams illustrating the schematic arrangement of certain elements of the bone graft injection systems of FIGS. 1A-E, respectively, in accordance with respective applications of the present invention;

FIGS. 3A-C are schematic illustrations of a portion of a composition delivery source of the bone graft injection systems of FIGS. 1A-C, in accordance with respective applications of the present invention;

FIGS. 3D-H are schematic illustrations of a portion of a composition delivery source of the bone graft injection systems of FIGS. 1D-E, in accordance with respective applications of the present invention;

FIGS. 4A-B are timelines schematically showing activation states of control circuitry of the bone graft injection system of FIGS. 1A-E, in accordance with respective applications of the present invention;

FIGS. 5A-D are schematic illustrations of the activation states of control circuitry of FIGS. 4A-B, in accordance with an application of the present invention;

FIGS. 6A-D are schematic illustrations of the activation states of control circuitry of FIGS. 4A-B, in accordance with an application of the present invention;

FIG. 7 is a schematic illustration of configurations of a mixing pump and a liquid-supply pump of the bone graft injection system of FIGS. 2A, 1B, and 1D, in accordance with an application of the present invention;

FIGS. 8A-B are schematic illustrations of a chamber of a composition delivery source of the bone graft injection system of FIG. 1A, in accordance with an application of the present invention;

FIG. 9 is a schematic illustration of a portion of a method of using the bone graft injection system of FIGS. 1A-F, in accordance with an application of the present invention;

FIG. 10 is a schematic illustration of a hydraulic sinus lift unit, in accordance with an application of the present invention;

FIGS. 11A-B are schematic illustrations of the hydraulic sinus lift unit of FIG. 10, with a fluid-delivery assembly thereof in unlocked and locked states, respectively, in accordance with an application of the present invention;

FIGS. 12A-B are schematic illustrations of a method of using the hydraulic sinus lift unit of FIG. 10 to perform a portion of a sinus lift procedure, in accordance with an application of the present invention;

FIG. 13 is a schematic illustration of a configuration of and method of using a shaft unit of a composition delivery source of the bone graft injection systems of FIGS. 1A-C, in accordance with an application of the present invention;

FIG. 14 is a schematic illustration of one use of the bone graft injection systems of FIGS. 1A-C for ridge augmentation, in accordance with an application of the present invention;

FIGS. 15A-B are schematic illustrations of one use of the bone graft injection systems of FIGS. 1A-C for performing a minimally-invasive spinal interbody fusion, in accordance with an application of the present invention; and

FIG. 16 is a schematic illustration of one use of the bone graft injection systems of FIGS. 1A-C for filling a bone defect, in accordance with an application of the present invention.

### DETAILED DESCRIPTION OF APPLICATIONS

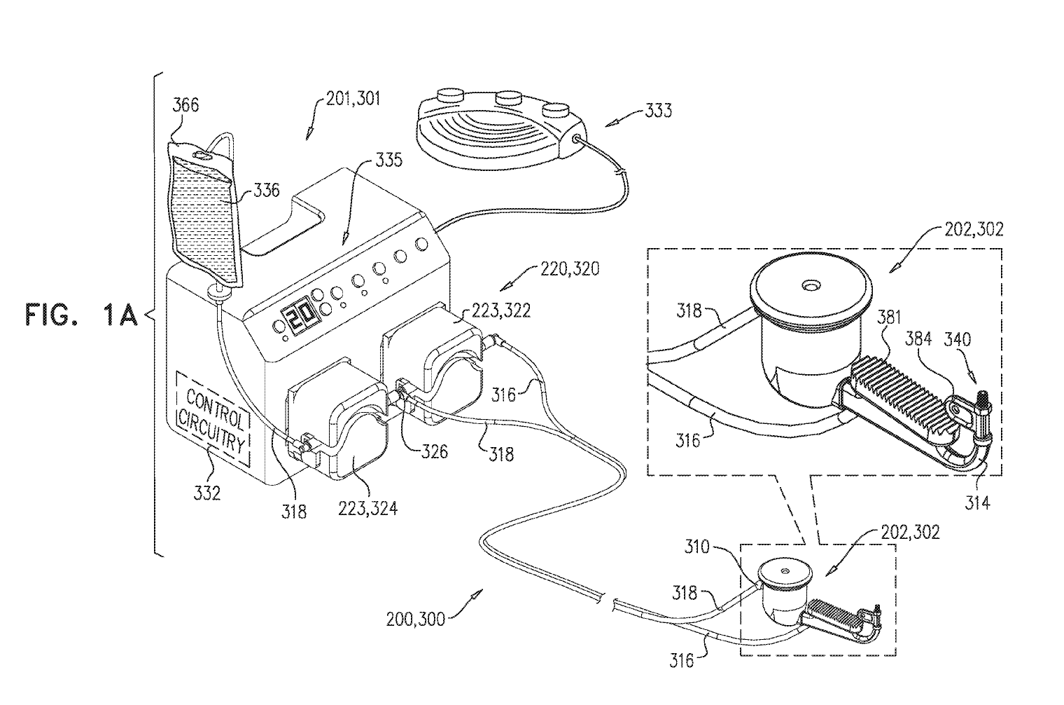

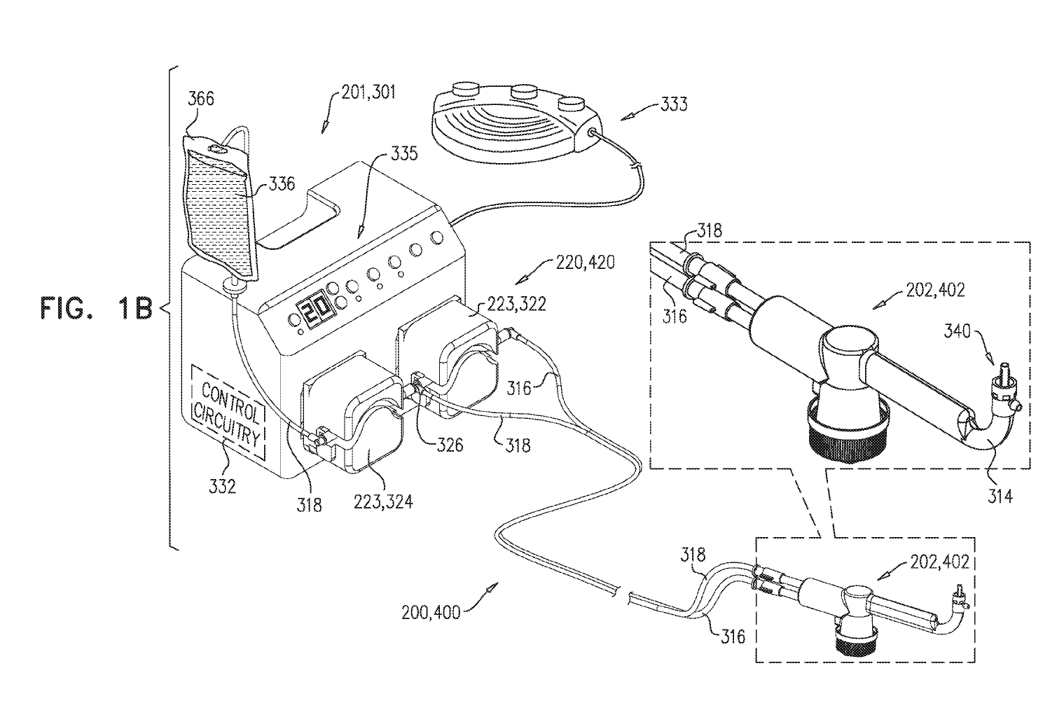

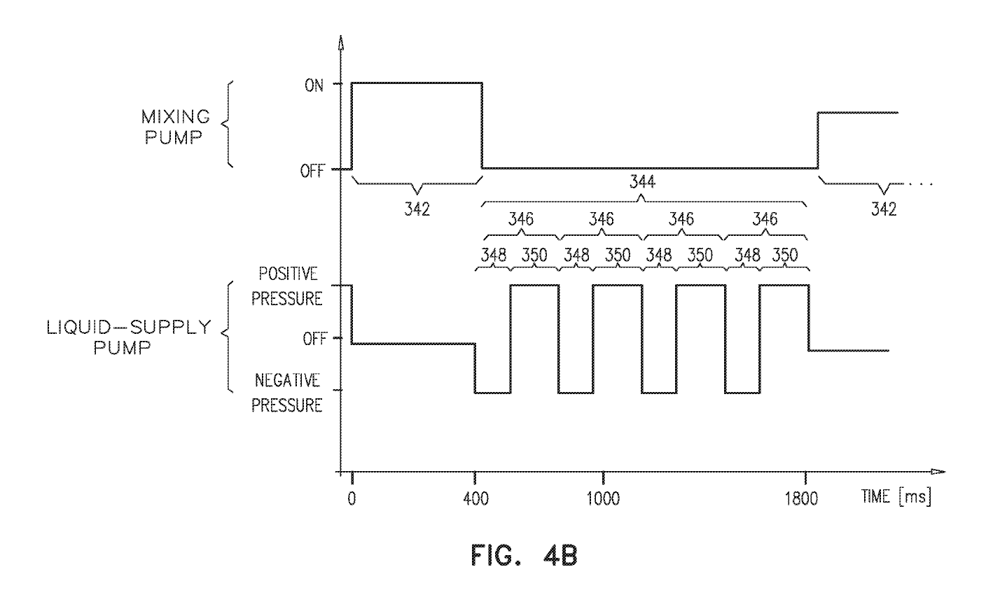

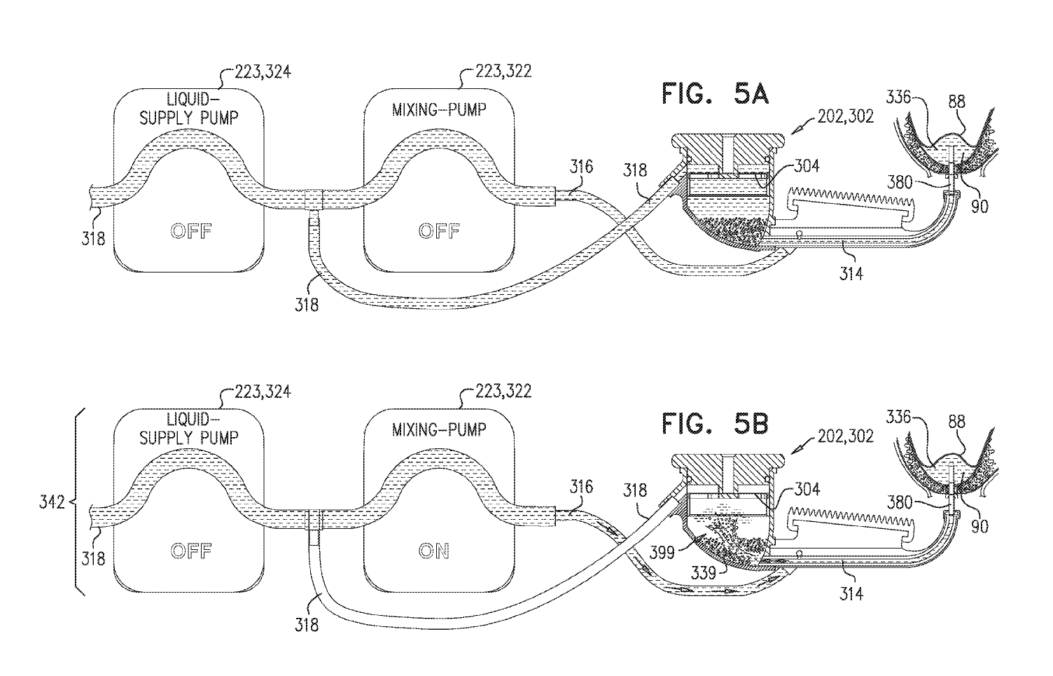

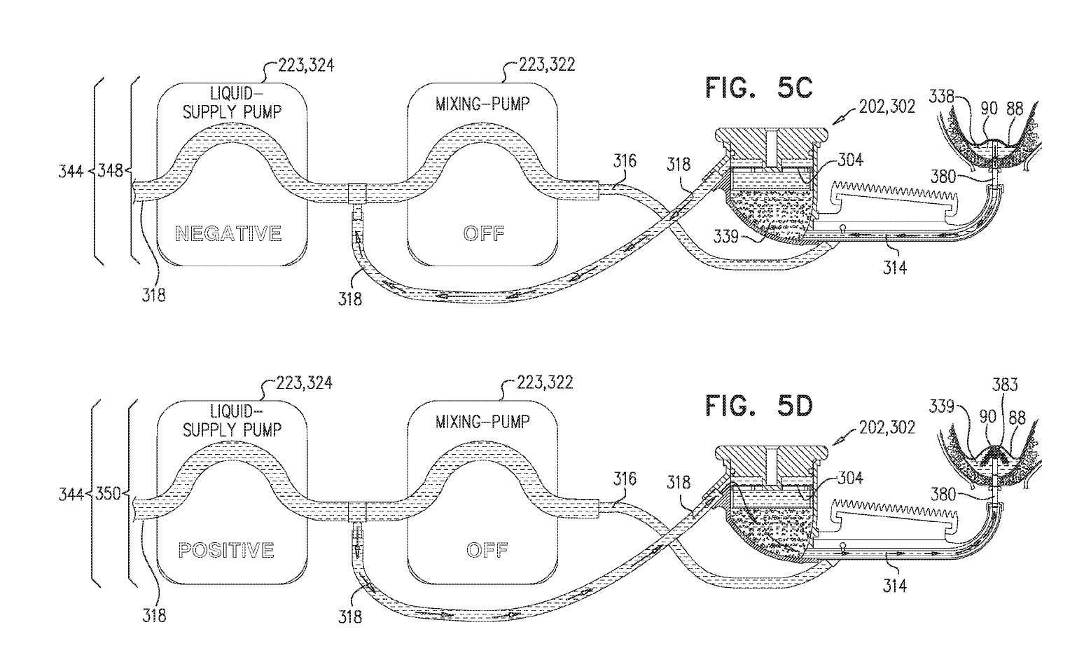

FIGS. 1A-E are schematic illustration of a bone graft injection system 220 for the insertion of solid particles, typically solid boric graft particles 334, into a cavity, in accordance with an application of the present invention. In FIGS. 1A, 1B, 1C, 1D, and 1E, bone graft injection system 220 comprises a bone graft injection system 320, a bone graft injection system 420, a bone graft injection system 520, a bone graft injection system 620, and a bone graft injection system 720, respectively. For example, the cavity may be a cavity 90 between a second side of a maxillary alveolar bone 82 and a Schneiderian membrane 88, shown in FIGS. 5A-D, 6A-D, 9, 12A-B, and 13, described hereinbelow. For some applications, boric graft injection system 220 is used to perform a minimally-invasive closed sinus lift surgical procedure for implanting a dental implant. The procedure is typically employed when a patient's maxillary alveolar bone 82 lacks sufficient bone mass to support a conventional dental implant. The procedure may be performed using any of the techniques described in the patents and patent application publications incorporated hereinbelow by reference, or using other sinus lift techniques known in the art.

For some applications, bone graft particles 334 comprise natural bone mineral particles (either xenograft or allograft), synthetic particles, demineralized bone matrix, an autograft, or bioactive, composites. For some applications, bone graft particles 334 have an average particle size (measured as the greatest dimension of each particle) of at least 0.01 mm, no more than 3 mm, and/or between 0.01 mm and 3 mm.

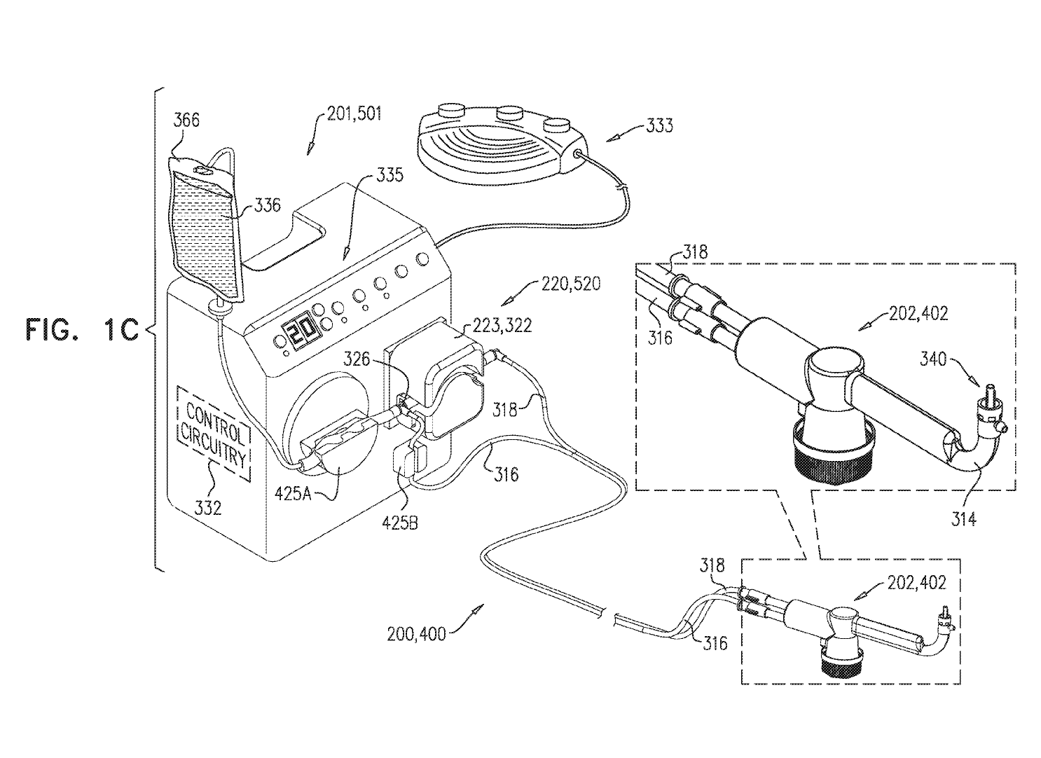

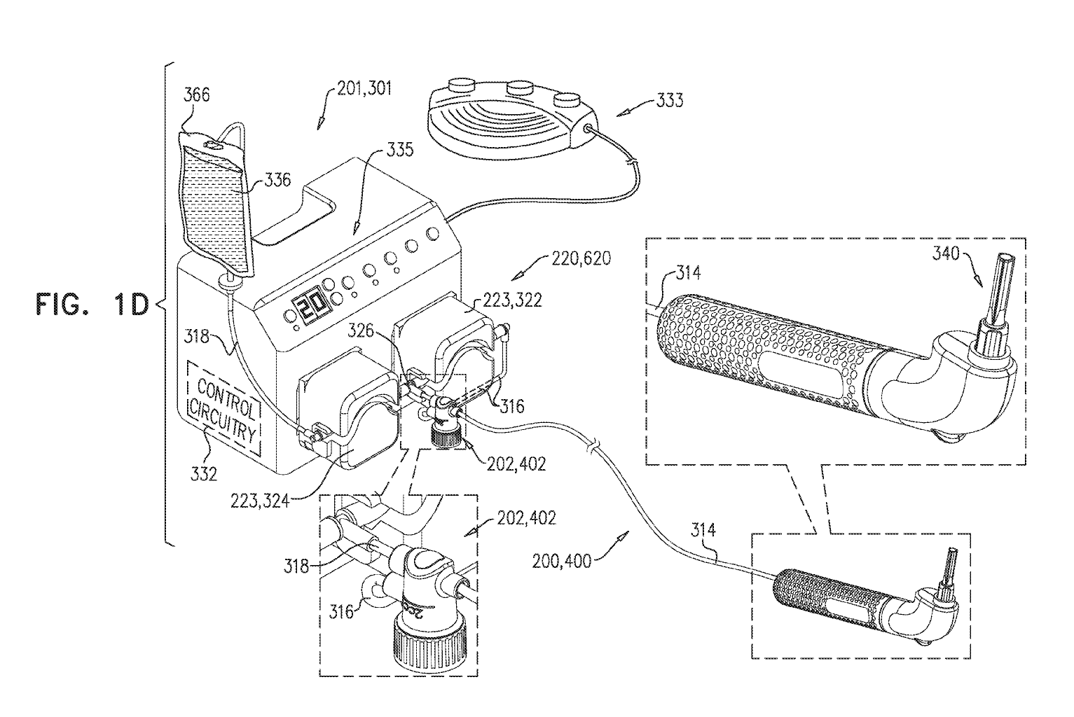

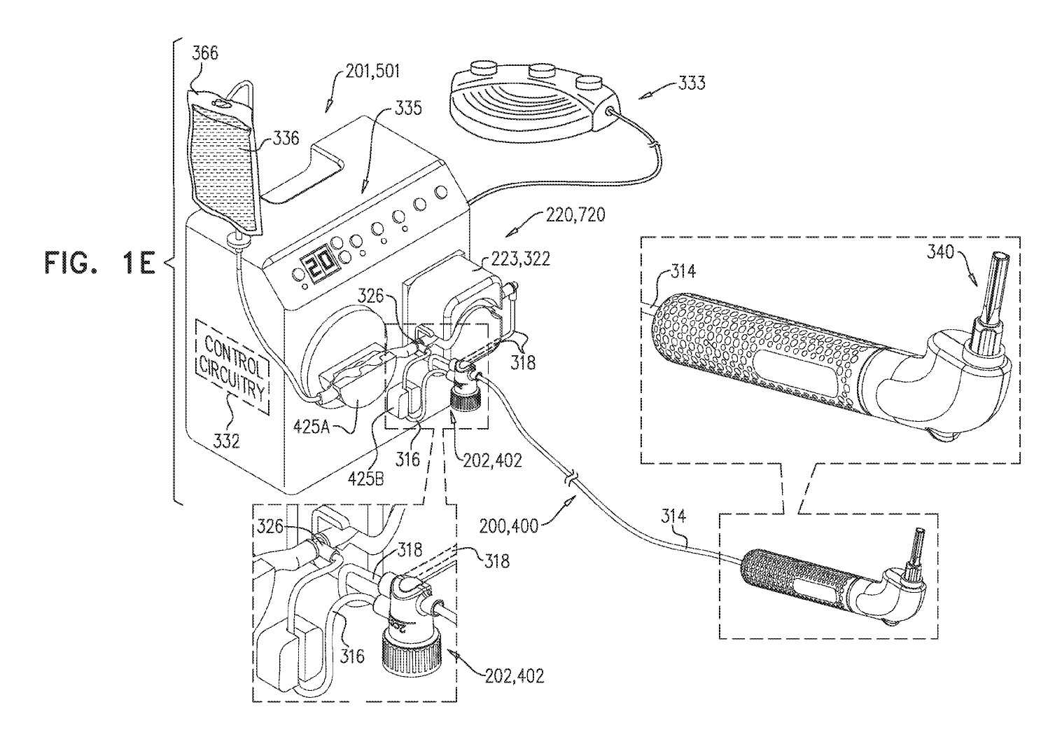

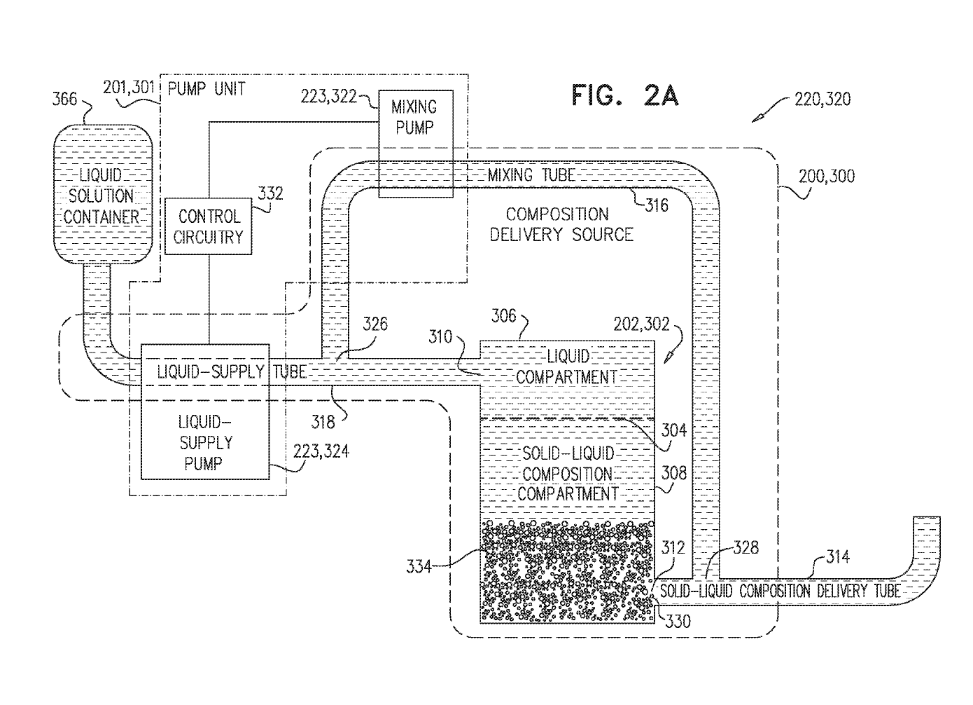

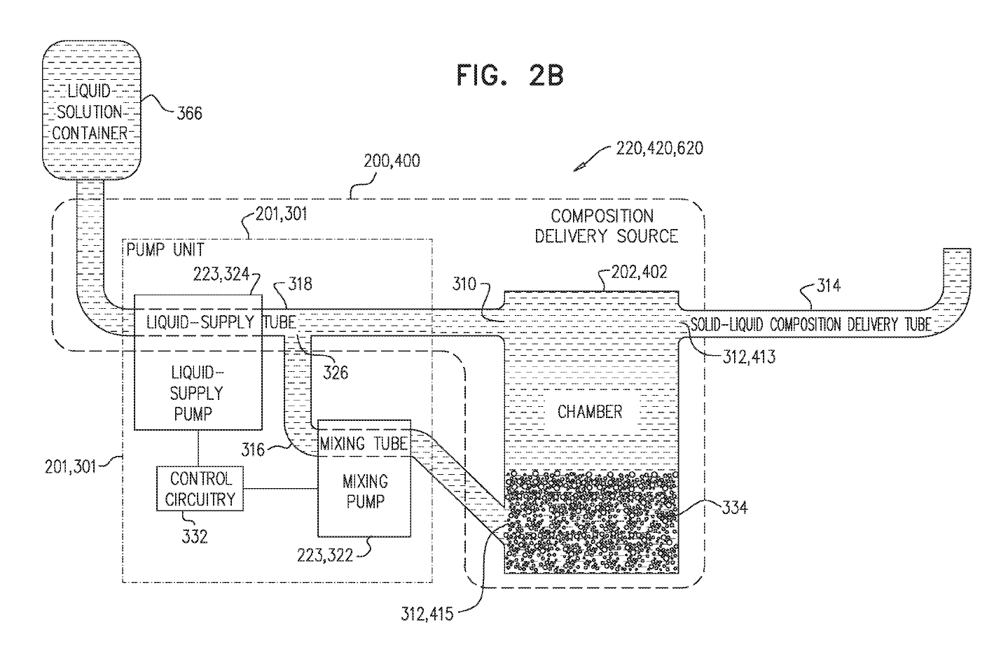

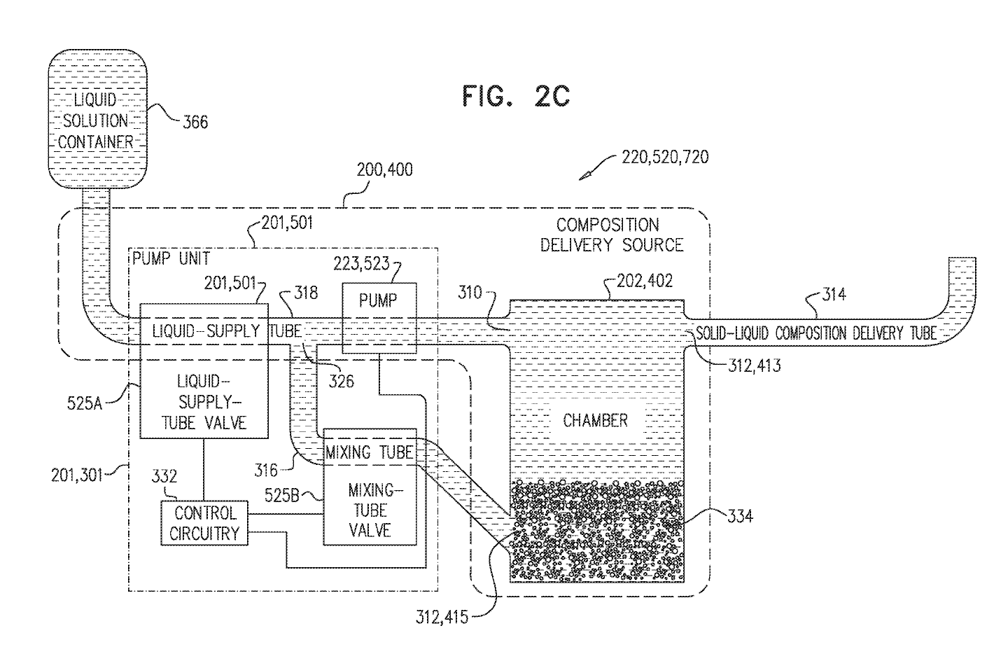

Reference is also made to FIGS. 2A-C, which are diagrams illustrating the schematic arrangement of certain elements of bone graft injection system 220, in accordance with respective applications of the present invention. FIG. 2A shows certain elements of bone graft injection system 320, FIG. 2B shows certain elements of bone graft injection systems 420 and 620, and FIG. 2C shows certain elements of bone graft injection systems 520 and 720. Bone graft injection system 220 is for use with a liquid solution container 366 containing a physiological liquid solution 336, such as saline solution or blood. Bone graft injection system 220 comprises a composition delivery source 200 and a pump unit 201. Composition delivery source 200 comprises a chamber 202.

Composition delivery source 200 of bone graft injection system 320 comprises a composition delivery source 300, and composition delivery source 200 of bone graft injection systems 420, 520, 620, and 720 comprises a composition delivery source 400. Chamber 202 of bone graft injection system 320 comprises a chamber 302, and chamber 202 of bone graft injection systems 420, 520, 620, and 720 comprises a chamber 402. Pump unit 201 of bone graft injection systems 320, 420, and 620 comprises a pump unit 301, and pump unit 201 of bone graft injection systems 520 and 720 comprises a pump unit 501. Composition delivery source 200 is typically configured to provide a solid-liquid composition 339 of bone graft particles 334 and physiological liquid solution 336. For some applications, solid-liquid composition 339 further comprises a radiopaque agent, to enable X-ray visualization of the procedure.

Typically, composition delivery source 200 is single-use and disposable, while pump unit 201 is reused many times. For some applications, the components of composition delivery source 200 are provided as a preassembled unit, while for other applications, one or more of the components are provided disconnected from one another and are assembled by a healthcare worker, for example, based on shape- or color-coding of the components. For some applications, chamber 202 is provided pre-loaded with solid bone graft particles 334, while for other applications, the user loads chamber 202 with solid bone graft particles 334 of his or her choice before or during the procedure. Pump unit 201 and composition delivery source 200 are configured to prevent incorrect coupling of composition delivery source 200 to pump unit 201.

Reference is made to FIGS. 1A-E and 2A-C. Pump unit 201 comprises one or more pumps 223, such as two pumps 322 and 324 (e.g., exactly two pumps), as in some configurations of pump unit 301, or exactly one pump 523, as in some configurations of pump unit 501. For some applications, the one or more pumps 223 are one or more peristaltic pumps, respectively.

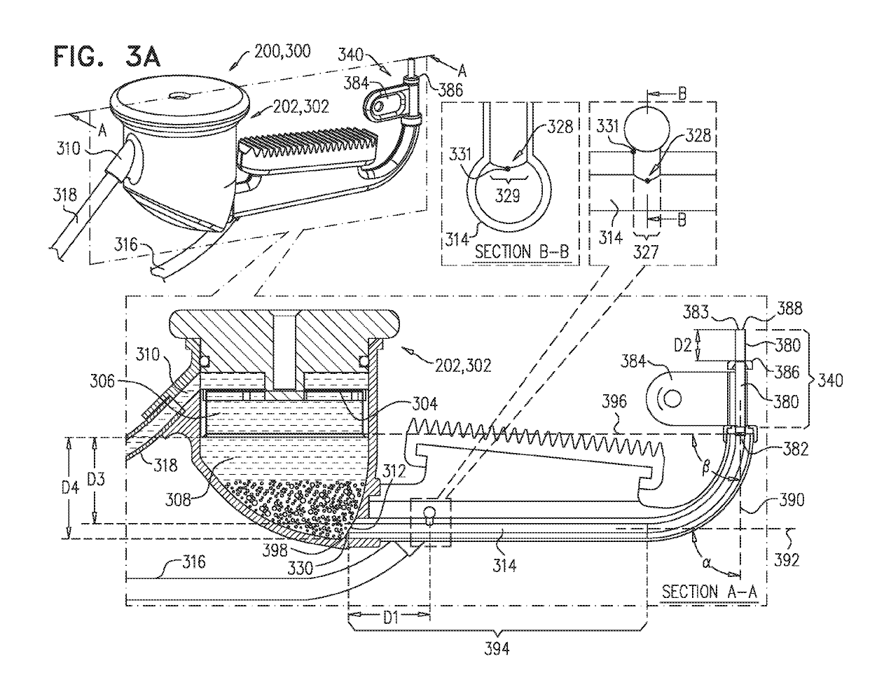

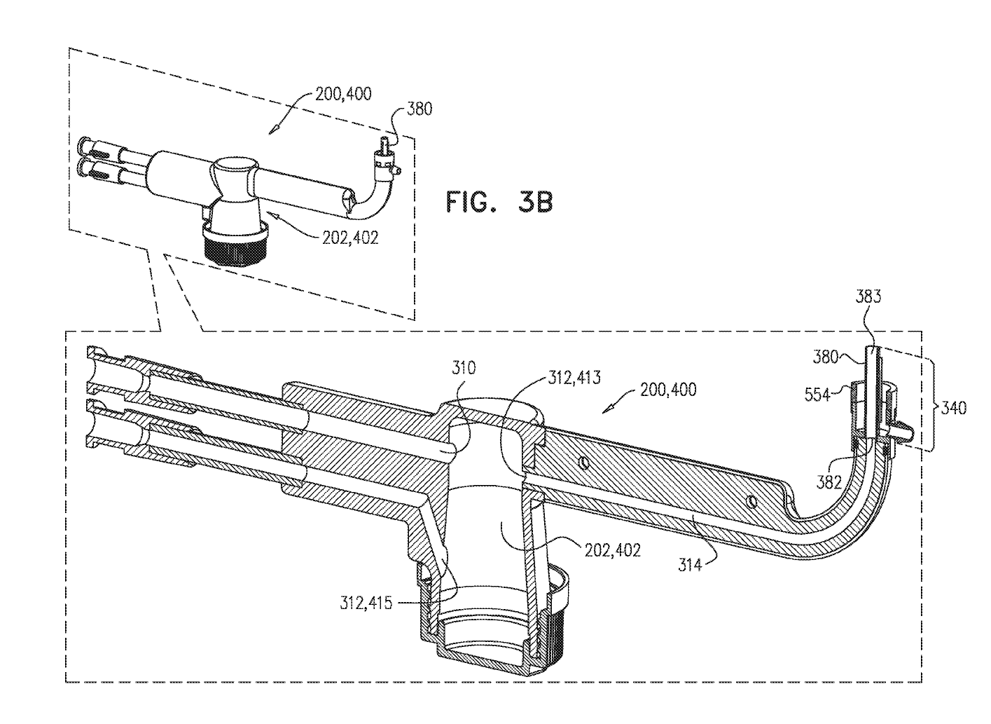

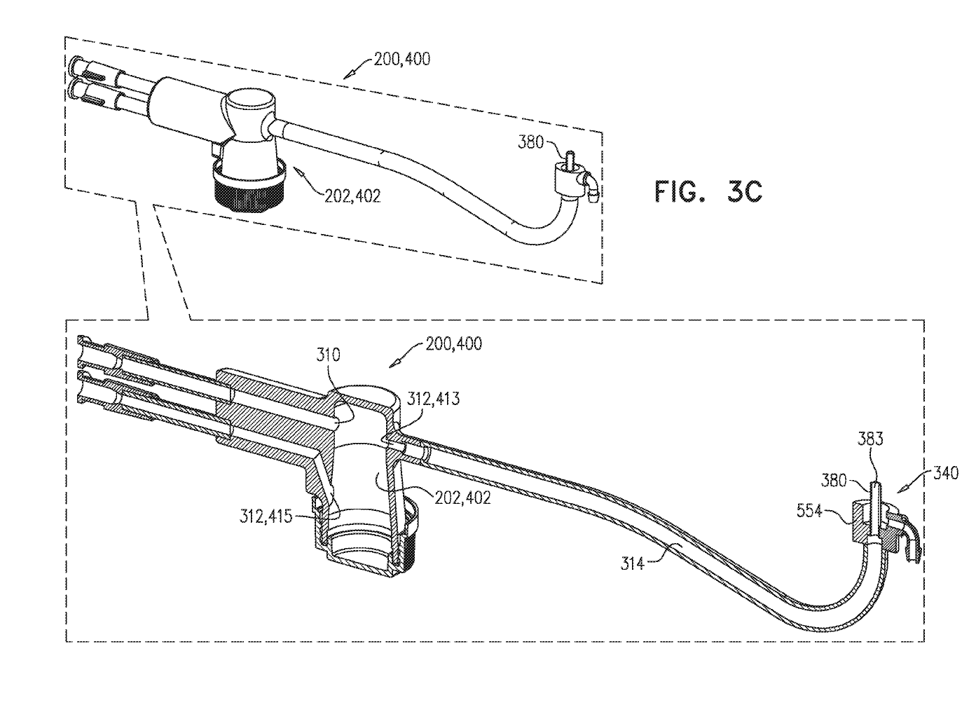

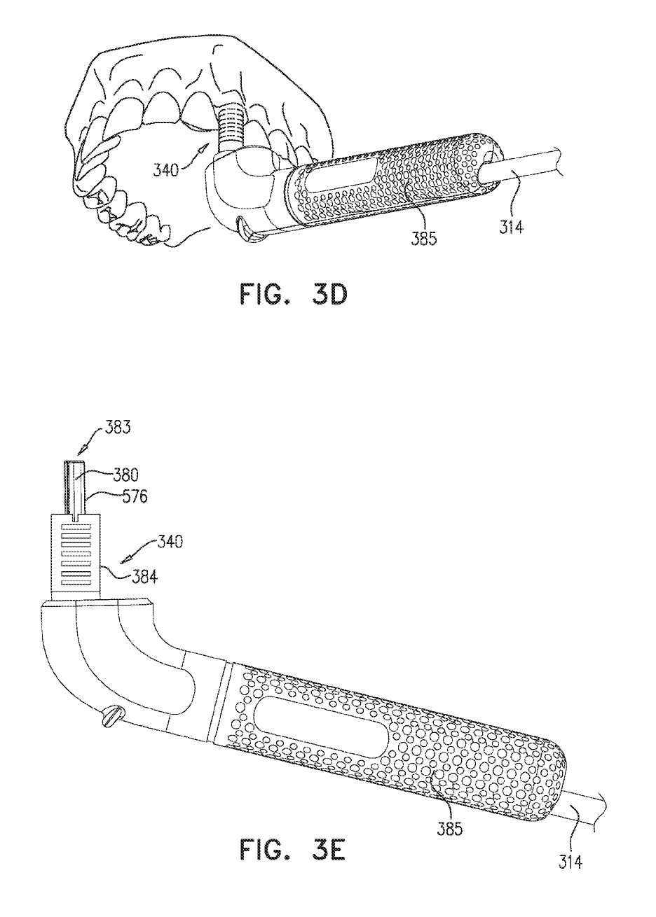

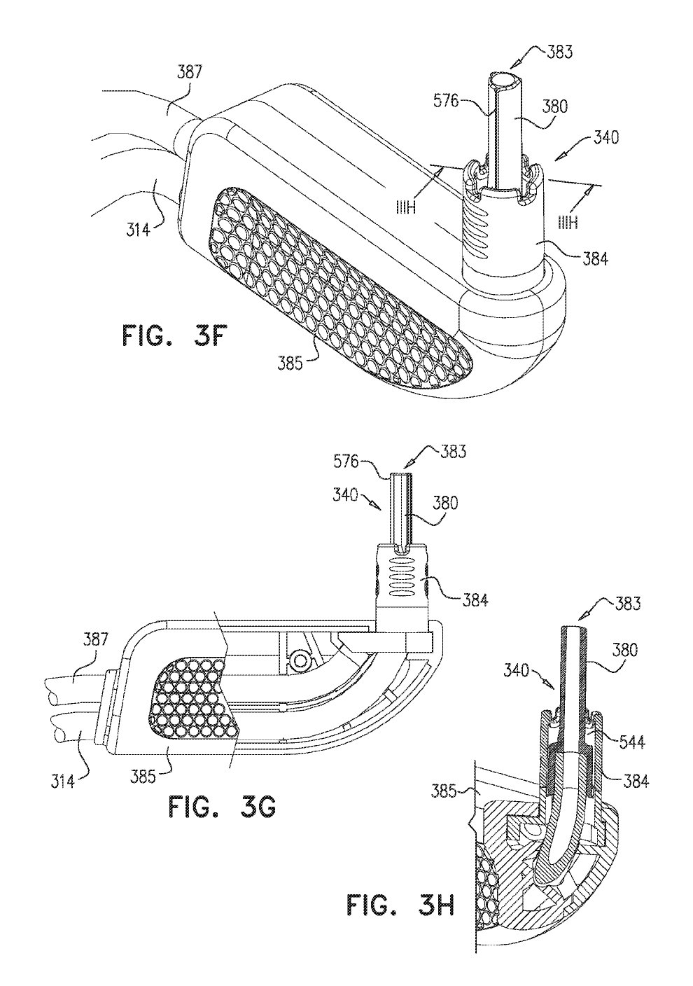

Reference is made to FIGS. 1A and 2A, and is additionally made to FIG. 3A, which is a schematic illustration of a portion of composition delivery source 300, in accordance with an application of the present invention. Composition delivery source 300 comprises chamber 302, which, in this configuration, optionally comprises a filter 304. Filter 304, if provided, is disposed within chamber 302 so as to divide chamber 302 into a liquid compartment 306 and a solid-liquid composition compartment 308. Typically, chamber 302 is shaped so as to define (a) one or more (e.g., exactly one) liquid ports 310 in fluid communication with an interior of chamber 302, typically with liquid compartment 306, and (B) one or more (e.g., exactly one) solid-liquid composition ports 312 in fluid communication with the interior of chamber 302, typically with solid-liquid composition compartment 308. Alternatively, chamber 302 is shaped so as to define exactly two solid-liquid composition ports 312, as in the configurations described hereinbelow with reference to FIGS. 1B-C, 2B-C, 3B-C, and 6A-D, mutatis mutandis. Alternatively, chamber 302 does not comprise filter 304 or any other internal filter, and thus defines a single compartment.

Composition delivery source 300 typically further comprises:
a solid-liquid composition delivery tube 314, which is in fluid communication with at least one of the one or more solid-liquid composition ports 312;
a mixing tube 316, which is in fluid communication with at least one of the one or more liquid ports 310 and at least one of the one or more solid-liquid composition ports 312; and
a liquid-supply tube 318, which is in fluid communication with at least one of the one or more liquid ports 310, and is coupled in fluid communication with an interior of liquid solution container 366.

Reference is made to FIGS. 1A, 1B, 1D, and 2A-B. In these configurations, pump unit 301 (and the one or more pumps 223 thereof) comprises:
a mixing pump 322, which is arranged to cause flow in mixing tube 316, typically unidirectionally; and
a liquid-supply pump 324, which is arranged to cause flow in liquid-supply tube 318, typically oscillating (bidirectional) flow.

It is noted that mixing tube 316 is considered to be in fluid communication with the at least one of the one or more liquid ports 310 and the at least one of the one or more solid-liquid composition ports 312 even though mixing tube 316 is intermittently not in such fluid communication because of the operation of liquid-supply pump 324, as described hereinbelow. Similarly, it is noted that liquid-supply tube 318 is considered to be in fluid communication with the at least one of the one or more liquid ports 310 and to be coupled in fluid communication with the interior of liquid solution container 366 even though liquid-supply tube 318 is intermittently not in such fluid communication because of the operation of mixing pump 322, as described hereinbelow.

For some applications, each of the tubes comprises one or more tube segments that are coupled together to form the complete tube, such as for applications in which the pumps do not comprise peristaltic pumps and respective tube segments are coupled to an inlet and an outlet of a pump.

For some applications, as shown in FIGS. 1A-C and 2A-C, mixing tube 316 (a) merges with liquid-supply tube 318 at an exit junction 326, and (b) is in fluid communication with the at least one of the one or more liquid ports 310 via a portion of liquid-supply tube 318. For other applications, liquid-supply tube 318 (a) merges with mixing tube 316 at an exit junction, and (b) is in fluid communication with the at least one of the one or more liquid ports 310 via a portion of mixing tube 316 (not shown, but functionally equivalent to the above-mentioned shown configuration).

For some applications, as shown in FIGS. 1A, 2A, and 3A, mixing tube 316 (a) merges with solid-liquid composition delivery tube 314 at a return junction 328, and (b) is in fluid communication with the at least one of the one or more solid-liquid composition ports 312 via a portion of solid-liquid composition delivery tube 314. This merging may help free any solid bone graft particles 334 that may become lodged in the one or more solid-liquid composition ports 312, because the flow into the one or more solid liquid composition ports 312 is via the portion of solid-liquid composition delivery tube 314 in the opposite direction of flow during delivery of solid-liquid composition 339 in particle-delivery activation state 344, as described hereinbelow with reference to 4A-B, 5A-D, and 6A-D.

For some applications, a proximal end 330 of solid-liquid composition delivery tube 314 is in fluid communication with the at least one of the one or more solid-liquid composition ports 312, and a distance D1 (labeled in FIG. 3A) between return junction 328 and proximal end 330 of solid-liquid composition delivery tube 314 is less than 60 mm, such as less than 20 mm. Disposing return junction 328 so close to proximal end 330 of solid-liquid composition delivery tube 314 reduces the amount of solid bone graft particles 334 pumped back from solid-liquid composition delivery tube 314 to solid-liquid composition compartment 308. For other applications, mixing tube 316 is in fluid communication with the at least one of the one or more solid-liquid composition ports 312 not via a portion of solid-liquid composition delivery tube 314. For some applications, an inner diameter of solid-liquid composition delivery tube 314 is at least 1.4 mm, no more than 1.8 mm, and/or between 1.4 and 1.8 mm. For some applications, solid-liquid composition delivery tube 314 is in fluid communication with exactly one of the one or more solid-liquid composition ports 312, and the exactly one port has a diameter of between 0.1 and 0.3 mm less than the inner diameter of solid-liquid composition delivery tube 314.

For some applications, an internal cross-sectional area of solid-liquid composition delivery tube 314 perpendicular to an axis of solid-liquid composition delivery tube 314 is non-decreasing from return junction 328 to a distal end of solid-liquid composition delivery tube 314. Typically, solid-liquid composition 339 does not flow along a converging flow path as it approaches the one or more solid-liquid composition ports 312 from solid-liquid composition compartment 308.

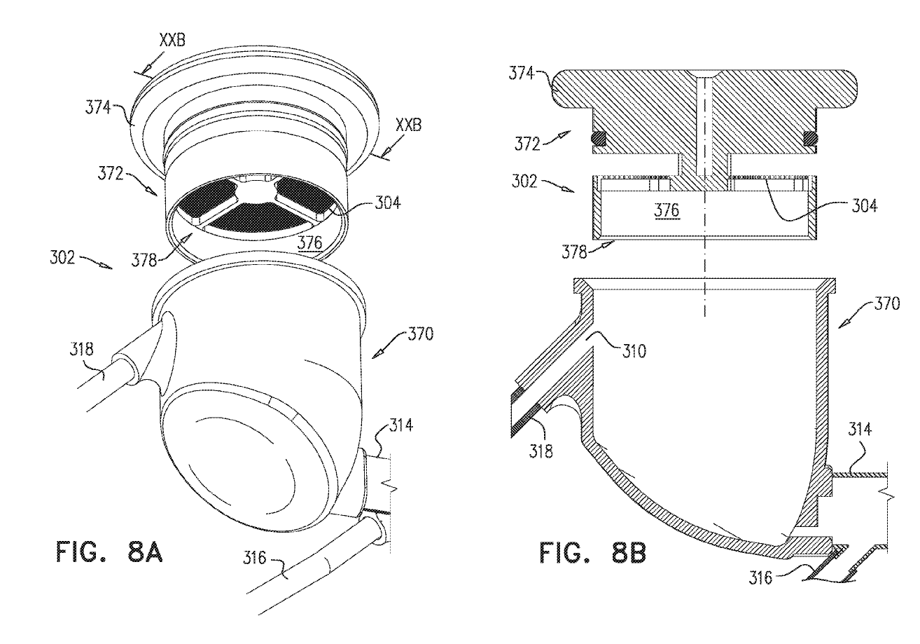

Reference is still made to FIG. 3A. For some applications, when chamber 302 is oriented upright in the operational position shown in FIG. 3A, return junction 328 is disposed on an upper side of the solid-liquid composition delivery tube 314. In other words, for some applications, return junction 328 is disposed along a longitudinal portion 327 of solid-liquid composition delivery tube 314 and around a circumferential portion 329 of solid-liquid composition delivery tube 314, and longitudinal portion 327 includes a point 331 that is closest to a cap 374 when cap 374 is coupled to receptacle component 370 (as described hereinbelow with reference to FIGS. 8A-B). Circumferential portion 329 includes point 331. This arrangement may reduce bone graft clogging, because solid bone graft particles 334, because of gravity, are less likely to flow upward back into mixing tube 316 toward mixing pump 322.

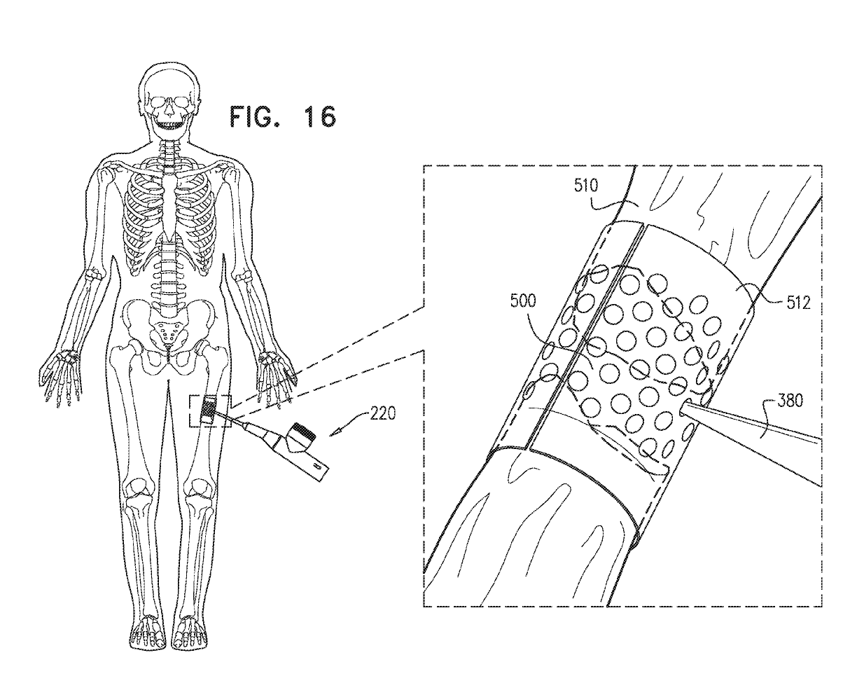

Reference is still made to FIG. 3A, and is additionally made to FIGS. 3B-H, which are schematic illustrations of a portion of composition delivery source 400, in accordance with respective applications of the present invention. For some applications, bone graft injection system 320 further comprises a shaft unit 340, which comprises a shaft delivery tube 380 in fluid communication with a distal end 382 of solid-liquid composition delivery tube 314. For some applications, shaft unit 340 is more rigid than at least a portion of solid-liquid composition delivery tube 314 (all or a portion of solid-liquid composition delivery tube 314 may be flexible). Shaft delivery tube 380 is further shaped so as to define a distal opening 383, which is typically disposed within 10 mm of a distal end 388 (labeled in FIG. 3A) of shaft delivery tube 380, such as within 5 mm of distal end 388, in fluid communication with shaft delivery tube 380. For example, distal opening 383 may be disposed at distal end 388, as shown in FIGS. 3A-C. Alternatively, for some applications, such as shown in FIG. 16B of International Application PCT/IL2016/050423, filed Apr. 20, 2016, which published as PCT Publication WO 2016/170540 and is assigned to the assignee of the present application and is incorporated herein by reference, shaft delivery tube 380 further comprises a cap disposed distal to distal opening 383; for these applications, distal opening 383 is typically disposed within 10 mm, e.g., within 5 mm, of distal end 388 of shaft delivery tube 380 (distal end 388 of shaft delivery tube 380 is defined by a distal-most point of the cap). For some applications, such as shown in FIGS. 3D-H, shaft unit 340 comprises a handle 385, which is coupled to shaft delivery tube 380 and solid-liquid composition delivery tube 314, which is typically coupled in fluid communication with shaft delivery tube 380 within handle 385.

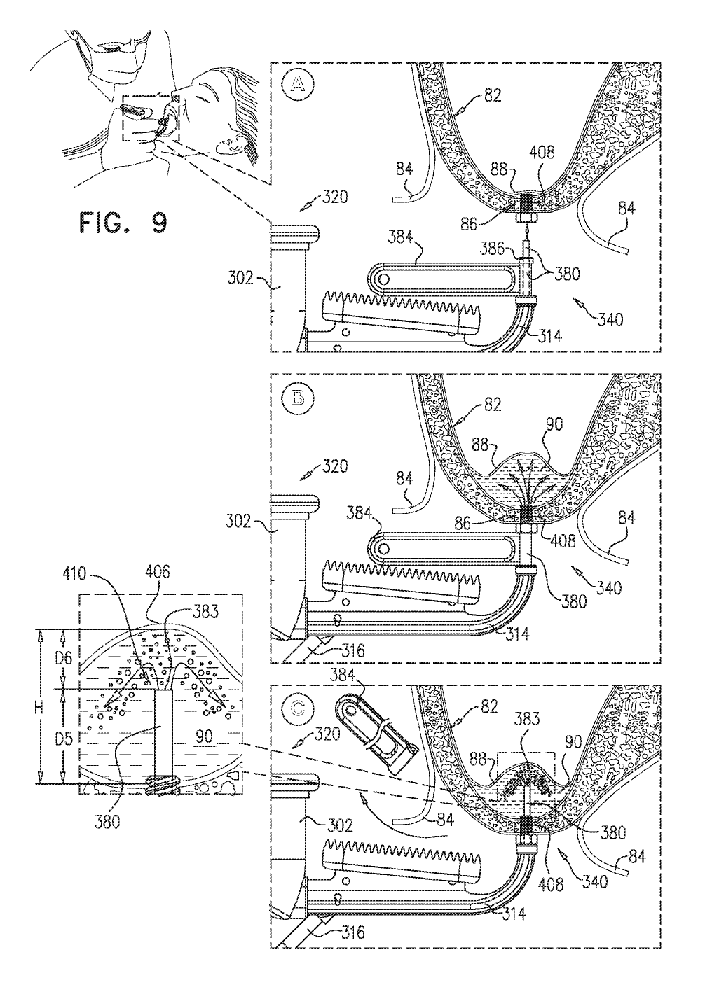

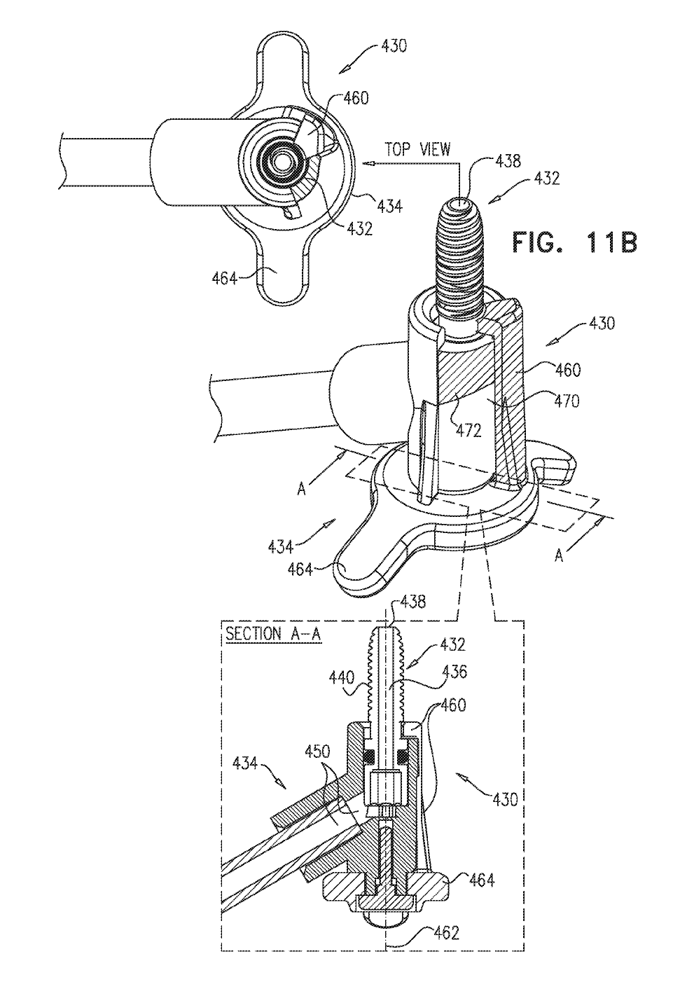

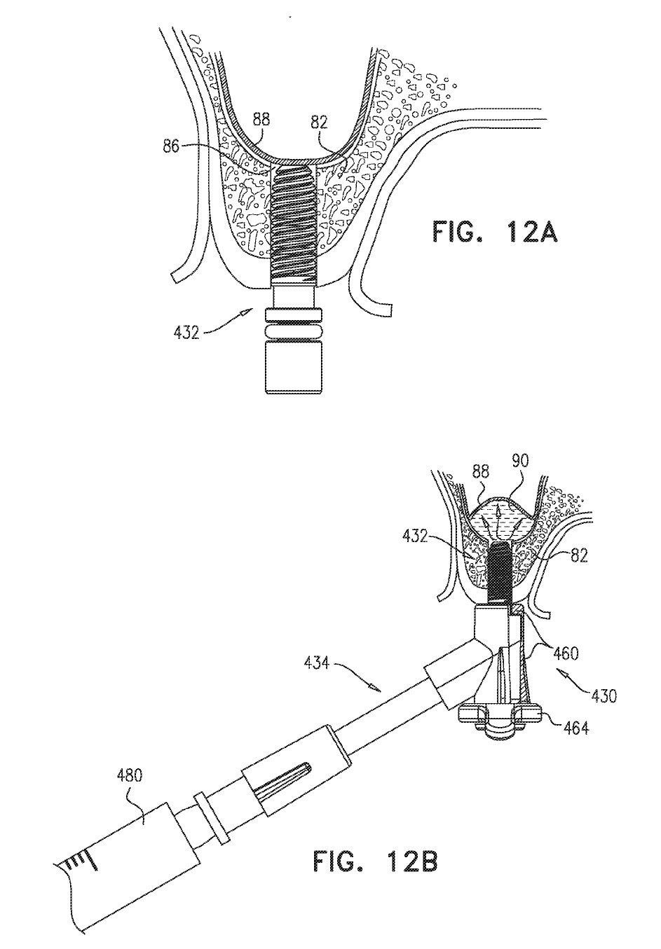

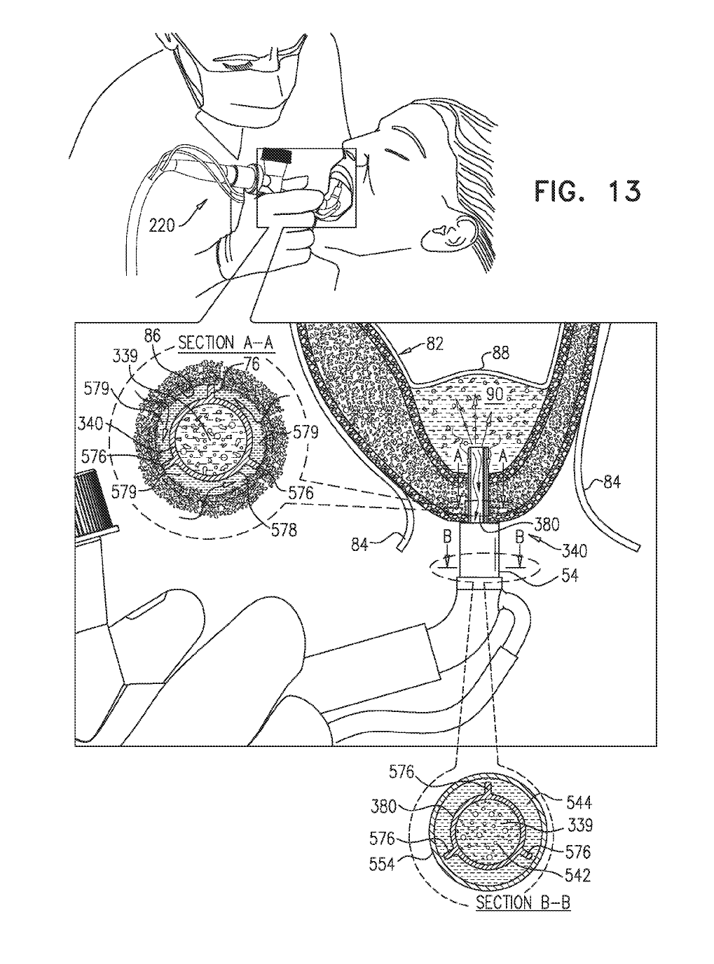

For some applications, such as shown in FIG. 3A and 3D-H, shaft unit 340 further comprises a removable depth limiting element 384, which is configured to limit a depth of insertion of shaft delivery tube 380 into a bore through a bone when shaft delivery tube 380 is inserted into the bore, such as described hereinbelow with reference to FIG. 9. For some applications, depth limiting element 384 has a length, measured alongside shaft delivery tube 380, of at least 6 mm, no more than 16 mm, and/or between 6 and 16 mm, such as at least 8 mm, no more than 12 mm, and/or between 8 and 12 mm. For some applications, a plurality of depth limiting elements 384 are provided having different respective lengths, and the surgeon selects the element with the appropriate length based on the patient's bone thickness. For some applications, such as shown in FIGS. 3D-H, depth limiting element 384 is shaped so as to define a portion of a drainage lumen 544 between at least a portion of an internal surface of depth limiting element 384 and a portion of an external surface of shaft delivery tube 380, such as described hereinbelow with reference to FIG. 13, mutatis mutandis.

For some applications, such as shown in FIGS. 3F-H, drainage lumen 544 is coupled in fluid communication with an optional suction tube 387, which in turn is coupleable to a conventional dental suction system. For other applications, such as shown in FIGS. 3D-E, suction tube 387 is not provided, and fluid that drains through drainage lumen 544 simply drips into the patient's mouth, and from there to conventional dental suction.

For some applications, such as shown in FIG. 3A, bone graft injection system 320 further comprises a soft bite surface 381, which is configured to provide a soft surface for the teeth to bite onto during a bone graft injection procedure. Typically, soft bite surface 381 faces in generally the same direction that shaft delivery tube 380 points.

For some applications, such as shown in FIG. 3A, shaft delivery tube 380 further comprises a sealing element 386 disposed around an external surface of shaft delivery tube 380, and configured to form a liquid-tight seal with (a) a channel of a screw, such as such as described hereinbelow with reference to FIG. 9, or (b) tissue around and outside the bore through the bone when shaft delivery tube 380 is inserted into the bore. Typically, depth limiting element 384 is removable from shaft unit 340 without removal of shaft unit 340 from sealing element 386. For some applications, distal end 388 of shaft delivery tube 380 is disposed more distally than sealing element 386 by a distance D2 of between 0 and 20 mm, e.g., between 3 and 15 mm.

Reference is still made to FIG. 3A. For some applications, shaft delivery tube 380 is straight (as shown in the figures). For some applications, when chamber 302, solid-liquid composition delivery tube 314, and shaft unit 340 are unconstrained, (a) central longitudinal axis 390 of shaft delivery tube 380 and (b) a central longitudinal axis 392 of a proximal longitudinal portion 394 of solid-liquid composition delivery tube 314 form an angle α (alpha) of between 70 and 110 degrees, such as between 85 and 95 degrees, e.g., 90 degrees. Typically, proximal longitudinal portion 394 of solid-liquid composition delivery tube 314 includes proximal end 330 of solid-liquid composition delivery tube 314. Alternatively or additionally, for some applications, when chamber 302, solid-liquid composition delivery tube 314, and shaft unit 340 are unconstrained, central longitudinal axis 390 of shaft delivery tube 380 and a plane 396 defined by filter 304 form an angle β (beta) of between 70 and 110 degrees, such as between 85 and 95 degrees, e.g., 90 degrees. Further alternatively or additionally, for some applications, when chamber 302 and solid-liquid composition delivery tube 314 are unconstrained, (a) central longitudinal axes 392 of proximal longitudinal portion 394 of solid-liquid composition delivery tube 314 and (b) plane 396 defined by filter 304 are parallel or form an angle of less than 20 degrees, e.g., less than 5 degrees. Typically, proximal longitudinal portion 394 of solid-liquid composition delivery tube 314 includes proximal end 330 of solid-liquid composition delivery tube 314.

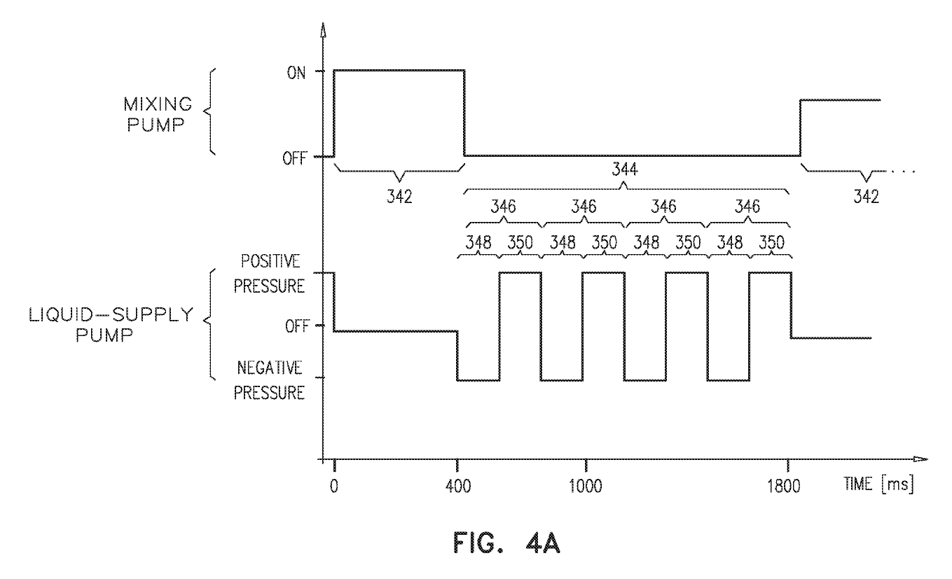

Reference is still made to FIG. 3A. For some applications, a closest distance D3 between the one or more solid-liquid composition ports 312 and filter 304 equals at least 5 mm, such as at least 10 mm, and/or is less than 50 mm. Alternatively or additionally, for some applications, the closest distance D3 between the one or more solid-liquid composition ports 312 and filter 304 equals at least 75% of a distance D4 between filter 304 and a point 398 on an interior of a wall or solid-liquid composition compartment 308 farthest from filter 304. These closest distances provide space for raising solid bone graft particles 334 in a puff 399 into physiological liquid solution 336, as described hereinbelow with reference to FIGS. 4A-B, 5A-D, and 6A-D. Typically, the one or more solid-liquid composition ports 312 are located through a side wall of solid-liquid composition compartment 308 (rather than a bottom wall of the solid-liquid composition compartment), to prevent clogging of the one or more solid-liquid composition ports 312 as the solid bone graft particles 334 settle after being raised.

Reference is made to FIGS. 1B-E, 2B-C, and FIGS. 3B-C. Composition delivery source 400 comprises chamber 402, which, in this configuration, typically does not comprise an internal filter, and thus typically defines a single compartment. Typically, chamber 402 is shaped so as to define (a) one or more (e.g., exactly one) liquid ports 310 in fluid communication with an interior of chamber 402, and (B) one or more (e.g., exactly one), such as two or more (e.g., exactly two) solid-liquid composition ports 312 in fluid communication with the interior of chamber 402. For some applications, the one or more solid-liquid composition ports 312 comprise (a) a solid-liquid composition delivery port 413 and (b) a solid-liquid composition inlet port 415. Alternatively chamber 402 is shaped so as to define exactly one solid-liquid composition port 312, as in the configuration described hereinabove with reference to FIGS. 1A, 2A, and 3A, mutatis mutandis.

Composition delivery source 400 further comprises:
solid-liquid composition delivery tube 314, which is in fluid communication with at least one of the one or more solid-liquid composition ports 312, such as with solid-liquid composition delivery port 413;
mixing tube 316, which is in fluid communication with at least one of the one or more liquid ports 310 and at least one of the one or more solid-liquid composition ports 312, such as with solid-liquid composition inlet port 415; and
liquid-supply tube 318, which is in fluid communication with at least one of the one or more liquid ports 310, and is coupled in fluid communication with the interior of chamber 402.

Reference is still made to FIGS. 1B-E, 2B-C, and FIGS. 3B-C. For some applications, when chamber 402 is oriented upright in the operational position shown in these figures:
the one or more liquid ports 310 are disposed near toe top of chamber 402, e.g., within 7 mm of the top of the chamber,
solid-liquid composition delivery port 413 is disposed near the lop of chamber 402, e.g., within 7 mm of the top of the chamber (e.g., at approximately the same height as the one or more liquid ports 310, e.g., respective distances from the top of the chamber of solid-liquid composition delivery port 413 and the one or more liquid ports 310 are within 20% of one another), and/or
solid-liquid composition inlet port 415 is disposed near the bottom of chamber 402, e.g., within 12 mm of the bottom of the chamber, typically below a height of the one or more liquid ports 310 and/or solid-liquid composition delivery port 413.

For some applications, a method of using bone graft injection systems 420 and 520 comprises orienting chamber 402 upright with bone graft particles 334 resting at the bottom of the chamber (before solid bone graft particles 334 are raised in puff 399). For some applications, when chamber 402 is thus oriented (before solid bone graft particles 334 are raised in puff 399):
bone graft injection systems 420 and 520 have one or more of the characteristics described immediately above,
solid-liquid composition delivery port 413 is disposed above a level of bone graft particles 334 (which are resting at the bottom of the chamber),
the one or more liquid ports 310 are disposed above the level of bone graft particles 334 (which are resting at the bottom of the chamber), and/or
solid-liquid composition inlet port 415 is disposed below the level of bone graft particles 334 or slightly above the level of bone graft particles 334.

Reference is made to FIGS. 3B and 3C. These configurations are generally similar, except that in the configuration shown in FIG. 3C, solid-liquid composition delivery tube 314 is more flexible and longer than in the configuration shown in FIG. 3B.

Reference is made to FIGS. 1B-E. The configurations shown in FIGS. 1D and 1E are generally similar to the configurations shown in FIGS. 1B and 1C, respectively, except that in the configurations shown in FIGS. 1D and 1E:
solid-liquid composition delivery tube 314 is longer and/or more flexible than in the configurations shown in FIGS. 1B and 1C, and
chamber 202 is closer to pump unit 201 than in the configurations shown in FIGS. 1B and 1C, and thus mixing tube 316 and liquid-supply tube 318 are substantially shorter than in the configurations shown in FIGS. 1B and 1C.

For some applications, it may be preferable to have the chamber situated at the pump unit, stationary and in the correct orientation independent of the orientation of the hone to which the bone graft is to be injected.

For some applications, a length of solid-liquid composition delivery tube 314 equals:
at least 300%, e.g., at least 500% or at least 750%, of a length of mixing tube 316,
at least 300%, e.g., at least 500% or at least 750%, of a length of liquid-supply tube 318.
at least 150%, e.g., at least 300% or at least 450%, of a sum of the length of mixing tube 316 and the length of liquid-supply tube 318, and/or
at least 50 cm, e.g., at least 100 cm.

For some applications, it may be preferable to manipulate the handle 385 at the end of a flexible tube or tube without having to support the weight of chamber 202,

For some applications, solid-liquid composition delivery tube 314 is configured to limit a radius of bending thereof (yet maintain acceptable flexibility), and/or inhibit kinking of the tube, which may prevent accumulation of particles and blockage. For example, solid-liquid composition delivery tube 314 may be have a relatively thick wall, may be provide with a sheath or coil therearound, and/or may have a structure wall,

Features of the configurations shown in FIGS. 1D and 1E may be implemented in combination with any of the other configurations described herein.

Reference is made to FIGS. 1A-E and 2A-C. For some applications, pump unit 201 further comprises control circuitry 332. Typically, pump unit 201 further comprises a power supply, electronics, a user interface 335 for controlling bone graft injection system 220, and/or a foot control 333 for controlling pump unit 201. For other applications, pump unit 201 does not necessarily comprise any circuitry, and the rotation and relative timing of rotation of the pump(s) are achieved mechanically (i.e., non-electronically), e.g., by connecting both pumps to a common axle, in configurations in which two pumps are provided.

Reference is now made to FIGS. 4A-B, which are timelines schematically showing activation states of control circuitry 332, in accordance with respective applications of the present invention. Reference is also made to FIGS. 5A-D, which are schematic illustrations of the activation states of control circuitry 332, in accordance with an application of the present invention, and to FIGS. 6A-D, which are schematic illustrations of the activation states of control circuitry 332, in accordance with another application of the present invention. For some applications, the activations states shown in FIGS. 5A-D are used with bone graft injection system 320, described hereinabove with reference to FIGS. 1A and 2A; bone graft injection system 420, described hereinabove with reference to FIGS. 1B and 2B; or bone graft injection system 620, described hereinabove with reference to FIGS. 1D and 2B. For some applications, the activations states shown in FIGS. 6A-D are used with bone graft injection system 520, described hereinabove with reference to FIGS. 1C and 2C, or bone graft injection system 720, described hereinabove with reference to FIGS. 1E and 2C. Although the phases are shown as rectangular in FIGS. 4A-B, they may instead be sloped, e.g., trapezoidal.

In some applications of the present application, bone graft injection system 220 is configured to repeatedly (a) mix solid bone graft particles 334 and physiological liquid solution 336 in chamber 202 (e.g., in solid-liquid composition compartment 308 in configurations in which the chamber defines the solid-liquid composition compartment) to form a solid-liquid composition 339 and (b) pump solid-liquid composition 339 into cavity 90 under a membrane, such as a Schneiderian membrane 88. For some applications, in order to perform the mixing, bone graft injection system 220 pumps filtered liquid from liquid compartment 306 into a lower portion (e.g., the bottom) of chamber 202 (e.g., of solid-liquid composition compartment 308 of chamber 302, in configurations in which the chamber defines the solid-liquid composition compartment), which raises solid boric graft particles 334 in a puff 399 into physiological liquid solution 336 higher in the chamber (e.g., solid-liquid composition compartment 308 of chamber 302). Because volume in chamber 202 is conserved as fluid is pumped out of the chamber (e.g., out of liquid compartment 306), the pumped fluid reenters chamber 302 (rather than entering the portion of solid-liquid composition delivery tube 314 beyond return junction 328 in the opposite direction of chamber 202 (to the right in FIGS. 2A-C, 3A-C, 5A-D, and 6A-D)).

Typically, this mixing is repeated periodically, because solid bone graft particles 334 very quickly settle and separate from physiological liquid solution 336 (generally nearly all of the particles settle within 500 ms). Typically, the immediately following particle delivery activation state 344 occurs (a) before most of solid bone graft particles 334 settle and separate from physiological liquid solution 336 and/or (b) even after solid bone graft particles 334 have settled (in which case typically the solid bone graft particles 334 that settled near the one or more solid-liquid composition ports 312, and/or bone graft particles puffed by the pulsating transfer itself).

FIGS. 5A and 6A show solid bone graft particles 334 settled at the bottom of chamber 202 before being mixed (in FIG. 5A, in solid-liquid composition compartment 308 of chamber 302, and in FIG. 6A, in chamber 402). This state occurs at the beginning of a bone graft injection performed with bone graft injection systems 320, 420, and 520, and occurs, at least approximately, near (e.g., slightly before, at, or slightly after) the end of each particle-delivery activation state 344, which is described below.

For some applications, in order to perform the mixing and pumping described immediately above, control circuitry 332 is configured to repeatedly (typically, in a plurality of cycles):
assume a mixing activation state 342, as shown in FIGS. 5B and 6B, in which control circuitry 332 mixes solid bone graft particles 334 and physiological liquid solution 336 in chamber 202 to form solid-liquid composition 339, by pumping physiological liquid solution 336 through mixing tube 316 and into chamber 202 (typically, the pumped physiological liquid solution was already disposed in mixing tube 316, and originated from chamber 202 via the one or more liquid ports 310), and
assume a particle-delivery activation state 344, as shown in FIGS. 5C-D and 6C-D; control circuitry 332, during at least a portion of particle-delivery activation state 344 (e.g., during positive particle-delivery activation sub-state 350, shown in FIGS. 5D and 6D, and described hereinbelow), applies positive pressure to pump solid-liquid composition 339 from chamber 202 into solid-liquid composition delivery tube 314.

Typically, in order to perform the mixing during mixing activation state 342, pump unit 201 is arranged such that the physiological liquid solution pumped into chamber 202 raises solid bone graft particles 334 in a puff 399 into physiological liquid solution 336 in the chamber.

For some of these applications, as shown in FIGS. 5A-D, the one or more pumps 223 comprise:
mixing pump 322, which is arranged to cause the flow in mixing tube 316 during mixing activation state 342; and
liquid-supply pump 324, which is arranged to cause the flow in the liquid-supply tube 318 during the particle-delivery activation state 344.

For some applications, in order to perform the mixing and pumping described above, control circuitry 332 is configured to repeatedly (typically, in the plurality of cycles):
assume mixing activation state 342, as shown in FIG. 5B, in which control circuitry 332 activates mixing pump 322 to mix solid bone graft particles 334 and physiological liquid solution 336 in solid-liquid composition compartment 308 to form solid-liquid composition 339, by pumping physiological liquid solution 336 through mixing tube 316 and into solid-liquid composition compartment 308 (typically, the pumped physiological liquid solution was already disposed in mixing tube 316, and originated from liquid compartment 306 via the one or more liquid ports 310), and
assume a particle-delivery activation state 344, as shown in FIGS. 5C and 5D; control circuitry 332, during at least a portion of particle-delivery activation state 344 (e.g., during positive particle-delivery activation sub-state 350, shown in FIG. 5D, and described hereinbelow), activates liquid-supply pump 324 to apply positive pressure to pump solid-liquid composition 339 from solid-liquid composition compartment 308 into solid-liquid composition delivery tube 314.

Typically, in order to perform the mixing during mixing activation state 342, the physiological liquid solution pumped into solid-liquid composition compartment 308 raises solid bone graft particles 334 in a puff 399 into physiological liquid solution 336 in the compartment.

For some applications, control circuitry 332 is configured to, when in particle-delivery activation state 344, activate liquid-supply pump 324 to apply the positive pressure to pump physiological liquid solution 336 (a) from liquid solution container 366, (b) through liquid-supply tube 318, (c) into liquid compartment 306, (d) through filter 304, (e) into solid-liquid composition compartment 308, (f) from solid-liquid composition compartment 308, and (g) to solid-liquid composition delivery tube 314.

For others of these applications, as shown in FIGS. 6A-D (and FIG. 2C), the one or more pumps 223 comprise a pump 23 (e.g., exactly one pump 523) that is arranged to:
cause the flow in mixing tube 316 during mixing activation state, and
cause the flow in liquid-supply tube 318 during particle-delivery activation state 344.
   Alternatively, for some applications, the one or more pumps 223 comprise a plurality of pumps, e.g., arranged in series.

To this end, for some applications, in order to perform the mixing and pumping described above, control circuitry 332 is configured to repeatedly (typically, in the plurality of cycles):
assume mixing activation state 342, as shown in FIG. 6B, in which control circuitry 332 (a) activates pump 523 to mix solid bone graft particles 334 and physiological liquid solution 336 in chamber 202 (e.g., chamber 402) to form solid-liquid composition 339, by pumping physiological liquid solution 336 through mixing tube 316 and into the chamber, and
assume particle-delivery activation state 344, as shown in FIGS. 6C and 6D; control circuitry 332, during at least a portion of particle-delivery activation state 344 (e.g., during positive particle-delivery activation sub-state 350, shown in FIG. 6D, and described hereinbelow), activates pump 523 to apply positive pressure to pump solid-liquid composition 339 from chamber 202 (e.g., chamber 402) into solid-liquid composition delivery tube 314.

Typically, in order to perform the mixing during mixing activation state 342, the physiological liquid solution pumped into the chamber raises solid bone graft particles 334 in a puff 399 into physiological liquid solution 336 in the chamber.

For some applications, when control circuitry 332 assumes mixing activation state 342, as shown in FIG. 6B, control circuitry 332 activates pump 523 to pump in the same direction as shown in FIG. 6C (i.e., the “negative” direction).

In some of these applications, pump unit 501 further comprises one or more valves 525, which are arranged to regulate flow in liquid-supply tube 318 and in mixing tube 316. For some of these applications, the one or more valves 525 comprise:
a liquid-supply-tube valve 525A, which is arranged to regulate flow in liquid-supply tube 318; and
a mixing-tube valve 525B, which is arranged to regulate flow in mixing tube 316,

For some applications, control circuitry 332 is configured to:
during mixing activation state 342, close liquid-supply-tube valve 525A and open mixing-tube valve 525B, and
during particle-delivery activation state 344, open liquid-supply-tube valve 525A and close mixing-tube valve 525B.

(Although liquid-supply-tube valve 525A and mixing-tube valve 525B are shown closed in FIG. 6A, this is not necessarily the case, and is of little or no significance because pump 523 is off in this initial state.)

Reference is made to FIGS. 5A-D and 6A-D. As mentioned above, control circuitry 332 is typically configured to repeatedly, in a plurality of cycles, assume mixing activation state 342 and particle-delivery activation state 344. For some applications, control circuitry 332 is configured to repeatedly assume mixing activation state 342 and particle-delivery activation state 344 over a period time period having a duration of at least 30 seconds, no more than 600 seconds, and/or between 30 and 600 seconds.

Reference is made to FIGS. 4A-B, 5A-D, and 6A-D. For some applications, control circuitry 332 is configured to assume mixing activation state 342 and particle-delivery activation state 344 at non-overlapping times, such as illustrated in FIGS. 4A-B. For some applications, control circuitry 332 is configured to assume particle-delivery activation state 344 within 500 ms after completing mixing activation state 342, such as within 100 ms after completing mixing activation state 342, e.g., immediately after completing mixing activation state 342, as shown in FIGS. 4A-B.

Reference is still made to FIGS. 4A-B, 5A-D, and 6A-D. For some applications, control circuitry 332 is configured to repeatedly, in alternation, (a) assume mixing activation state 342 for between 100 and 1200 ms, such as between 200 and 800 ms, e.g., 400 ms, and (b) assume particle-delivery activation state 344. For some applications, control circuitry 332 is configured to repeatedly, in alternation, (a) assume mixing activation state 342 for between 100 and 1200 ms, and (b) assume particle-delivery activation state 344 for between 150 and 3000 ms, such as between 1000 and 2000 ms, e.g., 1400 ms.

Reference is still made to FIGS. 4A-B, 5A-D, and 6A-D. In some applications of the present invention, control circuitry 332 is configured, during each of one or more negative-positive particle delivery cycles 346 of particle-delivery activation state 344, to assume:
a negative particle-delivery activation sub-state 348, as shown in FIGS. 5C and 6C, in which control circuitry 332 activates liquid-supply pump 324 to apply negative pressure to pump liquid from solid-liquid composition delivery tube 314 toward chamber 202 (in FIG. 5C, toward liquid compartment 306 of chamber 302 via solid-liquid composition compartment 308 of chamber 302, and in FIG. 6C, toward chamber 402), and
a positive particle-delivery activation sub-state 350, as shown in FIGS. 5D and 6D, in which control circuitry 332 activates liquid-supply pump 324 to apply the positive pressure to pump solid-liquid composition 339 from chamber 202 into solid-liquid composition delivery tube 314 (in FIG. 5D, from solid-liquid composition compartment 308 of chamber 302, and in FIG. 6D, from chamber 402); a direction of pumping of liquid-supply pump 324 in positive particle-delivery activation sub-state 350 is opposite a direction of pumping of liquid-supply pump 324 in negative particle-delivery activation sub-state 348.

In other words, control circuitry 332 is configured to cause liquid-supply pump 324 to oscillate during each of one or more negative-positive particle delivery cycles 346. For some applications, control circuitry 332 is configured to deactivate liquid-supply pump 324 for a short period between the positive and negative particle-delivery activation sub-states.

Although negative particle-delivery activation sub-state 348 is illustrated in FIGS. 5C and 6C as preceding positive particle-delivery activation sub-state 350, shown in FIGS. 5D and 6D, this is not necessarily the case. Indeed, particularly for applications using the configuration of shaft unit 340 described hereinabove with reference to FIGS. 3D-H and/or hereinbelow with reference to FIG. 13, control circuitry 332 is typically configured to begin particle-delivery activation state 344 in each of the particle-delivery-state cycles with positive particle-delivery activation sub-state 350, such as described hereinabove.

It is noted that even though chamber 402 typically does not comprise an internal filter, flow of solid-liquid composition 339 does not generally reach pump 523 during negative particle-delivery activation sub-state 348, because the duration of negative particle-delivery activation sub-state 348 is not long enough for the flow of solid-liquid composition 339 to reach pump 523.

During positive particle-delivery activation sub-state 350, solid-liquid composition 339 is injected into cavity 90. Solid bone graft particles 334 of solid-liquid composition 339 typically quickly settle toward the bottom of cavity 90 (generally within 100 ms). As a result, physiological liquid solution 336, substantially without solid bone graft particles 334, remains near distal opening 383 of shaft delivery tube 380. During the immediately following negative particle-delivery activation sub-state 348, mostly this physiological liquid solution 336 remaining near distal opening 383, rather than the settled solid bone graft particles 334, is pumped back into solid-liquid composition delivery tube 314. This non-return of solid bone graft particles 334 may be aided by positioning distal opening 383 near the roof of cavity 90, as described hereinbelow with reference to blow-up C of FIG. 9. Thus each positive-negative cycle results in a net delivery of solid bone graft particles 334 to cavity 90.

For some applications, at least a portion of solid-liquid composition 339 that is pumped out of chamber 202 in a given positive particle-delivery activation sub-state 350 exits distal opening 383 into cavity 90 before the completion of the given positive particle-delivery activation sub-state 350, such as at least 50%, e.g., at least 80%, such as 100%. For some applications, control circuitry 332 is configured to pump, throughout positive particle-delivery activation sub-state 350, a volume of solid-liquid composition 339 that is greater than a combined volume of solid-liquid composition delivery tube 314 and shaft delivery tube 380, such as equal to at least 100% of the combined volume, and/or less than 700% of the combined volume.

For some applications, control circuitry 332 is configured to assume particle-delivery activation state 344 in a plurality of particle-delivery-state cycles, and to begin particle-delivery activation state 344 in each of the particle-delivery-state cycles with negative particle-delivery activation sub-state 348. Beginning with the negative particle-delivery activation sub-state 348 reduces the risk of accidentally overfilling cavity 90 with solid-liquid composition 339, which might burst Schneiderian membrane 88. For other applications, particularly those using the configuration of shaft unit 340 described hereinabove with reference to FIGS. 3D-H and/or hereinbelow with reference to FIG. 13, control circuitry 332 is configured to begin particle-delivery activation state 344 in each of the particle-delivery-state cycles with positive particle-delivery activation sub-state 350. The drainage of excess physiological liquid solution 336 into drainage lumen 544 substantially eliminates the risk of accidentally overfilling cavity 90 with solid-liquid composition 339.

For some applications, as mentioned above, control circuitry 332 is configured to assume mixing activation state 342 and particle-delivery activation state 344 at non-overlapping times.

For some applications, control circuitry 332 is configured to provide a plurality of the negative-positive particle delivery cycles 346 during particle-delivery activation state 344. For some applications, control circuitry 332 is configured to provide up to 10 of the negative-positive particle delivery cycles 346 during particle-delivery activation state 344, such as between 3 and 6 cycles 346, e.g., 4 cycles 346.

For some applications, control circuitry 332 is configured to assume negative particle-delivery activation sub-state 348 for between 25 and 300 ms, such as between 100 and 200 ms, 175 ms, during each of the one or more negative-positive particle delivery cycles 346. For some applications, control circuitry 332 is configured to assume negative particle-delivery activation sub-state 348 for between 25 and 100 ms during each of the one or more negative-positive particle delivery cycles 346.

For some applications, control circuitry 332 is configured to assume positive particle-delivery activation sub-state 350 for between 25 and 300 ms, such as between 100 and 200 ms, e.g., 175 ms, during each of the one or more negative-positive particle delivery cycles 346. For some applications, control circuitry 332 is configured to assume positive particle-delivery activation sub-state 350 for between 25 and 100 ms during, each of the one or more negative-positive particle delivery cycles 346.

For some applications, control circuitry 332 is configured to assume negative particle-delivery activation sub-state 348 for between 25 and 300 ms during each of the one or more negative-positive particle delivery cycles 346, and to assume positive particle-delivery activation sub-state 350 for between 25 and 300 ms during each of the one or more negative-positive particle delivery cycles 346.

For some applications, control circuitry 332 is configured to assume negative particle-delivery activation sub-state 348 for a first duration during each of the one or more negative-positive particle delivery cycles 346, and to assume positive particle-delivery activation sub-state 350 for a second duration during each of the one or more negative-positive particle delivery cycles 346, the second duration equal to between 80% and 120% of the first duration, such as between 90% and 110% of the first duration, e.g., 100% of the first duration, such as shown in FIG. 4A. For other applications, particularly those using the configuration of shaft unit 340 described hereinabove with reference to FIGS. 3D-H and/or hereinbelow with reference to FIG. 13, the second duration is greater than 100% of the first duration, such as equal to at least 110%, e.g., between 110% and 200%, of the first duration. The excess physiological liquid solution 336 drains into drainage lumen 544, such as described hereinabove with reference to FIGS. 3D-H and/or hereinbelow with reference to FIG. 13.

For some applications, in the configuration shown in FIGS. 5A-D, control circuitry 332 is configured to, when in negative particle-delivery activation sub-state 348, activate liquid-supply pump 324 to pump the liquid from solid-liquid composition delivery tube 314, into solid-liquid composition compartment 308, and into liquid compartment 306.

For some applications, control circuitry 332 is configured, during each of one or more particle delivery cycles of particle-delivery activation state 344, to assume:
a positive particle-delivery activation sub-state, in which control circuitry 332 activates liquid-supply pump 324 to apply the positive pressure to pump solid-liquid composition 339 from chamber 202 into solid-liquid composition delivery tube 314 (in FIG. 5D, from solid-liquid composition compartment 308 of chamber 302, and in FIG. 6D, from chamber 402), and
a neutral particle-delivery activation sub-state, in which control circuitry 332 deactivates liquid-supply pump 324, and physiological liquid solution 336 is allowed to drain by gravity into the oral cavity, such as described hereinabove with reference to FIGS. 3D-E, or by suction provided by a separate dental suction system, such as described hereinabove with reference to FIGS. 3F-H.

For some applications, control circuitry 332 is configured, during each of one or more particle delivery cycles of particle-delivery activation state 344, to assume:
a strong positive particle-delivery activation sub-state, in which control circuitry 332 activates liquid-supply pump 324 to apply the positive pressure at a strong positive pressure, to pump solid-liquid composition 339 from chamber 202 into solid-liquid composition delivery tube 314, and
a weak positive particle-delivery activation sub-state, in which control circuitry 332 activates liquid-supply pump 324 to apply the positive pressure at a weak positive pressure (weaker than the strong positive pressure mentioned above), to pump solid-liquid composition 339 from chamber 202 into solid-liquid composition delivery tube 314.

For some applications, control circuitry 332 is configured to assume the weak positive particle-delivery activation sub-state for a first duration during each of the one or more particle delivery cycles, and to assume the strong positive particle-delivery activation sub-state for a second duration during each of the one or more particle delivery cycles, the second duration less than the first duration, e.g., between 50% and 90% of the first duration. For some applications, the weak positive particle-delivery activation sub-state is replaced by deactivation.

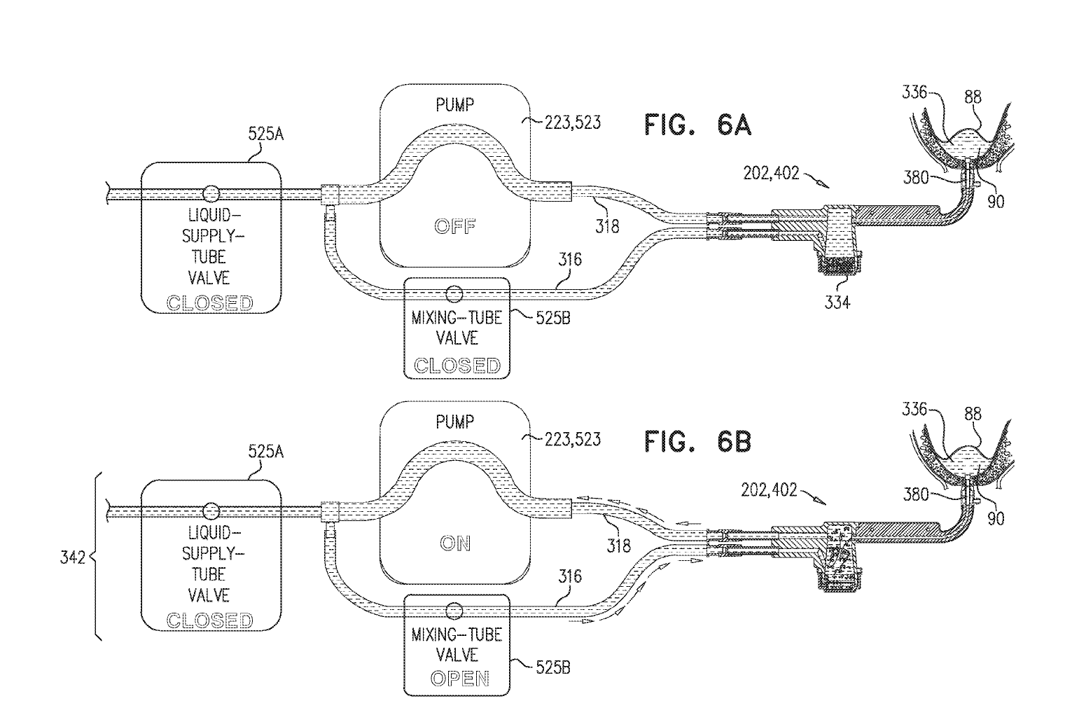

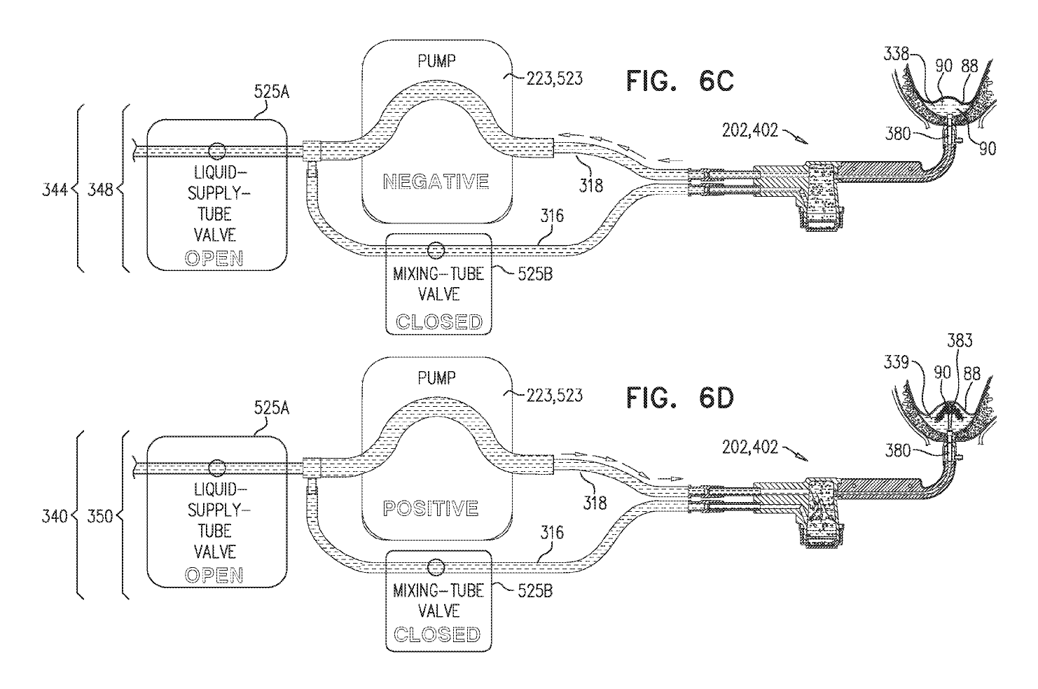

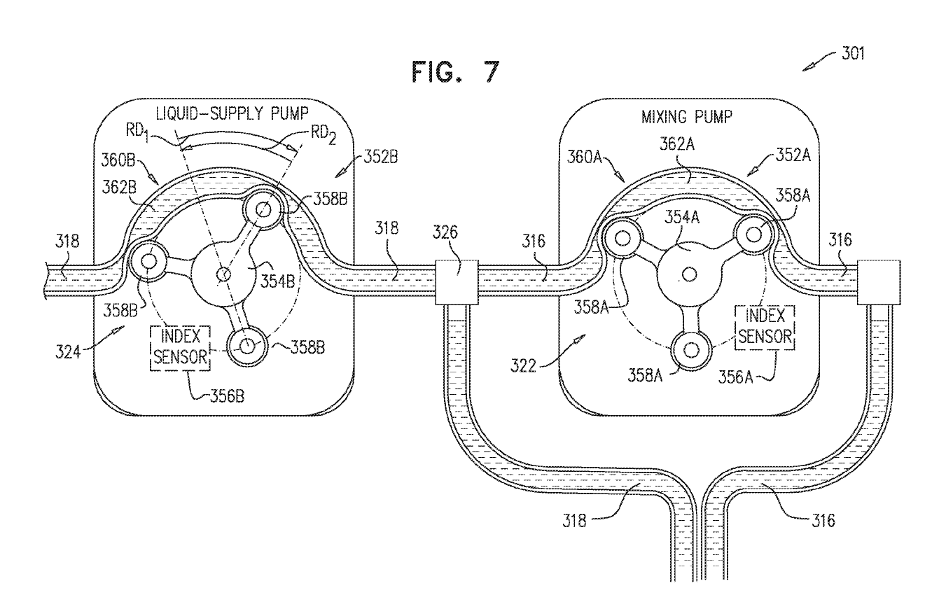

Reference is made to FIG. 7, which is a schematic illustration of configurations of mixing pump 322 and liquid-supply pump 324, in accordance with an application of the present invention, in these configurations, mixing pump 322 is a mixing peristaltic pump 352A, and liquid-supply pump 324 is a liquid-supply peristaltic pump 352B. Peristaltic pumps 352A and 352B comprise (a) respective rotors 354A and 354B, (b) respective motors, and, for some applications, (c) respective index sensors 356A and 356B, which identify respective rotational positions of rotors 354A and 354B. Mixing peristaltic pump 352A comprises one or more rollers 358A (typically, three or more rollers 358A, such as exactly three rollers 358A), and liquid-supply peristaltic pump 352B comprises one or more rollers 358B (typically, two or more rollers 358, such as three or more rollers 358B, such as exactly three rollers 358B). For some applications, the index sensors comprise optical sensors; for example, the rollers may comprise visible flags that serve as indices, and the optical sensors may image the flags to ascertain the rotational positions of the rollers and thus the rotors. Alternatively, for some applications, the index sensors comprise position (rotation) sensors. FIG. 7 shows mixing and liquid-supply peristaltic pumps 352A and 352B in exemplary respective starting rotational positions within respective rotational cycles.

Mixing peristaltic pump 352A comprises a pump casing 360A that is shaped so as to define a partial-circle mixing tube channel 362A in which a portion of mixing tube 316 is disposed. Similarly, liquid-supply peristaltic pump 352B comprises a pump casing 360B that is shaped so as to define a partial-circle liquid-supply tube channel 362B in which a portion of liquid-supply tube 318 is disposed. For some applications, the portions of the tubes disposed in the partial-circle liquid-supply tube channels comprise silicone, which may be more flexible than the material that other portions of the tubes comprise. Alternatively or additionally, for some applications, the portions of the tubes disposed in the partial-circle liquid-supply tube channels may have larger diameters than the diameters of the other portions of the tubes. These larger diameters may increase the pumping rate. The smaller diameters of the other portions of the tubes may reduce the total volume of fluid in the system, which may reduce the volume of fluid needed to operate the system. Typically, mixing peristaltic pump 352A rotates unidirectionally, clockwise in FIG. 7.

For some applications, mixing peristaltic pump 352A and the portion of mixing tube 316 disposed within mixing tube channel 362A are configured such that mixing peristaltic pump 352A pumps at least 2 cc, no more than 4 cc, and/or between 2 and 4 cc of fluid per full revolution, such as 2.7 cc. For some of these applications, the portion of mixing tube 316 disposed within mixing tube channel 362A has an inner diameter of at least 3.2 mm, no more than 9.6 mm, and/or between 3.2 and 9.6 mm, e.g., 6.4 mm.

For some applications, liquid-supply peristaltic pump 352B and the portion of liquid-supply tube 318 disposed within liquid-supply tube channel 362B are configured such that liquid-supply peristaltic pump 352B pumps at least 2 cc, no more than 4 cc, and/or between 2 and 4 cc of fluid per full revolution, such as 2.7 cc. For some of these applications, the portion of liquid-supply tube 318 disposed within liquid-supply tube channel 362B has an inner diameter of at least 3.2 mm, no more than 9.6 mm, and/or between 3.2 and 9.6 mm, e.g., 6.4 mm.

When a roller 358 is fully engaged and closes off a tube, the roller pushes a certain amount of liquid as it rotates. As the leading roller begins to disengage from the tube, the next roller behind the leading roller continues the pushing. However, since the leading roller is disengaging from the tube, the leading roller allows the tube to open up and hold a larger volume of liquid. This absorption of liquid not pushed out of the pump reduces flow. There are no voids anywhere in the tube. A reverse effect occurs as the next roller begins engaging the tube. Maximum flow is achieved during the period in which the leading roller is fully engaged with tube. This is the range in which the oscillating liquid-supply peristaltic pump 352B works. In a closed system, such as described herein, the amount of liquid in the pillows in liquid-supply peristaltic pump 352B is minimal when the most rollers are engaged with the tube. If exactly three rollers are provided, this minimum occurs, for example, when two of the rollers are symmetrically located at 10 o'clock and 2 o'clock. For some applications, this is the starting rotational position of mixing pump peristaltic pump 352A, since maximum liquid is in cavity 90 under Schneiderian membrane 88.

Liquid-supply peristaltic pump 352B is capable of (a) pumping fluid at an average rate throughout a full 360-degree revolution of rotor 354B at a certain speed, and (b) pumping fluid at a maximum rate during portions of the full 360-degree revolution at the certain speed. The maximum rate is greater than the average rate. For some applications, control circuitry 332 is configured, when in both positive and negative particle-delivery activation sub-states 350 and 348, to activate liquid-supply peristaltic pump 352B to (a) rotate rotor 354B, at the certain speed, a partial revolution equal to a fraction of the full 360-degree revolution of rotor 354B, the fraction less than 1, and (b) pump, throughout the partial revolution, the fluid at the maximum rate.

For some applications, control circuitry 332 is configured:
when in positive particle-delivery activation sub-state 350, to activate liquid-supply peristaltic pump 352B to rotate the rotor 354B, in a first rotational direction RD₁ (e.g., clockwise in FIG. 7), a first partial revolution equal to a fraction of a full 360-degree revolution of the rotor 354B, the fraction less than 1, and
when in negative particle-delivery activation sub-state 348, to activate liquid-supply peristaltic pump 352B to rotate rotor 354B, in a second rotational direction RD₂ (e.g., counterclockwise in FIG. 7) opposite the first rotational direction RD₁, a second partial revolution equal to the fraction of the full 360-degree revolution of the rotor.

This technique for rotating rotor 354B results in liquid-supply peristaltic pump 352B producing a net output of zero, while maximizing both the positive and negative flow, because one of rollers 358B is always squeezing, and thus occluding, liquid-supply tube 318 (and thus pumping).

For some applications, control circuitry 332 is configured, throughout positive particle-delivery activation sub-state 350, to activate liquid-supply peristaltic pump 352B to:
rotate rotor 354B a partial revolution equal to a fraction of a full 360-degree revolution of rotor 354B, the fraction less than the quotient of 1 divided by the total number of rollers 358B, or, for example, less than or equal to the quotient of 0.5 divided by the total number of rollers 358B (for example, in FIG. 7, the fraction is indicated by arrow RD₁ and equals ⅙, which is the quotient of 0.5 divided by 3), and
pump, throughout the partial revolution, a volume of fluid that is greater than the product of the fraction and a volume of fluid pumpable throughout the full 360-degree revolution of the rotor.
   For some applications, in order to achieve this volume of fluid pumping, control circuitry 332 is configured to rotationally position rotor 354B such that a lead one of rollers 358B is rotationally aligned with (fully squeezing) mixing tube channel 362A (and is thus operative) throughout positive particle-delivery activation sub-state 350 (the lead roller is the forward-most roller rotationally aligned with partial-circle mixing tube channel 362A; one or more additional rollers may also be rotationally aligned with the tube channel, trailing the lead roller). For example, if the upstream entrance to mixing tube channel 362A is disposed at 9 o'clock and the downstream exit of mixing tube channel 362A is disposed at 3 o'clock (as shown in FIG. 7), the exactly one of rollers 358B may operate between 11 o'clock and 1 o'clock throughout positive particle-delivery activation sub-state 350.

As used in the present application, including in the claims, “throughout” a time period (e.g., a particular state or sub-state) means from the beginning to the end of the time period (e.g., an occurrence of the state or sub-state). As mentioned above, each of the states and sub-states typically occur a plurality of non-contiguous times during operation of bone graft injection system 320.

For some applications, mixing peristaltic pump 352A comprises a total number of rollers 358A equal to at least two, and control circuitry 332 is configured to assume mixing activation state 342 a plurality of times in alternation with particle-delivery activation states 344, and to begin mixing activation states 342 with rotor 354A at respective starting rotational positions, which are identical to one another or rotationally offset from one another by the product of (a) 360 degrees divided by the total number of rollers 358A and (b) a positive integer (i.e., 1 or greater). For example, for configurations in which mixing peristaltic pump 352A comprises exactly three rollers 358A, such as shown in FIG. 7, there are three starting rotational positions which result in the same flow rate over the same partial rotational cycle.

For some applications, mixing peristaltic pump 352A comprises an odd total number of rollers 358A, the odd total number equal to at least one (e.g., at least three), and control circuitry 332 is configured to assume mixing activation state 342 a plurality of times in alternation with particle-delivery activation states 344, and to begin each of mixing activation states 342 with an aligned total number of rollers 358A rotationally aligned with mixing tube channel 362A, the aligned total number equal to more than half of the odd total number. (Thus, in the case in which mixing peristaltic pump 352A comprises exactly three rollers 358A, as shown in FIG. 7, control circuitry 332 is configured to begin each of mixing activation states 342 with two of rollers 358A rotationally aligned with mixing tube channel 362A, i.e., the aligned total number equals 2, which is more than half of the odd total number (1.5).) As a result of this configuration, each of mixing activation states 342 begins with a minimum volume of liquid held within the portion of mixing tube 316 in mixing tube channel 362A. As a result, any rotation of rotor 354A will draw liquid from the system and therefore will, if anything, reduce the volume of liquid in cavity 90 under Schneiderian membrane 88, thereby avoiding accidental overfilling of cavity 90 and bursting of Schneiderian membrane 88. In addition, cavity 90 returns to its full and maximum-filled state at end of each of the mixing activation states 342. As a result, mixing peristaltic pump 352A has full control of the maximum volume and variation in volume in cavity 90. Typically, the mixing activation state always begins when the volume cavity 90 is at a maximum, in order to avoid overfilling the cavity and bursting Schneiderian membrane 88.

For some applications, control circuitry 332 is configured, when in mixing activation state 342, to rotate mixing peristaltic pump 352A between ⅓ and 3 revolutions, such as one revolution, such as for applications in which mixing peristaltic pump 352A comprises exactly three rollers 358A. More generally, for some applications, control circuitry 332 is configured, when in mixing activation state 342, to rotate mixing peristaltic pump 352A between (a) a number of revolutions and (b) 3 revolutions, the number of revolutions equal to the quotient of 1 divided by the number of rollers 358A. For some applications, control circuitry 332 is configured, when in mixing activation state 342, to rotate mixing peristaltic pump 352A at a rate of at least 50 rpm (revolutions per minute), no more than 600 rpm, and/or between 50 and 600 rpm, e.g., 150 rpm. This rapid rotation helps generate the puff 399 described hereinabove.

For some applications, control circuitry 332 is configured:
when in positive particle-delivery activation sub-state 350, to activate liquid-supply pump 324 to pump a volume of between 0.1 and 2 cc of fluid (e.g., between 0.2 and 0.9 cc, such as between 0.3 and 0.6 cc), and
when in negative particle-delivery activation sub-state 348, to activate liquid-supply pump 324 to pump the volume of fluid.

Alternatively, control circuitry 332 is configured, when in negative particle-delivery activation sub-state 348, to activate liquid-supply pump 324 to pump less than the volume of fluid pumped when in positive particle-delivery activation sub-state 350, such no more than 90% of the volume of fluid pumped when in positive particle-delivery activation sub-state 350.

Alternatively or additionally, for some applications, control circuitry 332 and mixing pump 322 are configured such that throughout mixing activation state 342 (i.e., during each occurrence of mixing activation state 342 in configurations in which mixing activation state 342 occurs more than once in alternation with particle-delivery activation state 344), mixing pump 322 pumps between 0.5 and 9 cc of physiological liquid solution 336, such as between 1.8 and 3.9 cc of physiological liquid solution 336.

For some applications, control circuitry 332 is configured to assume particle-delivery activation state 344 a plurality of times in alternation with mixing activation states 342, and to begin each of particle-delivery activation states 344 with rotor 354B at a same rotational position.

For some applications, control circuitry 332 and liquid-supply pump 324 are configured such that during at least a portion of positive particle-delivery activation sub-state 350, liquid-supply pump 324 pumps physiological liquid solution 336 at a rate of at least 3 cc/sec, such as at least 7 cc/sec. Alternatively or additionally, for some applications, control circuitry 332 and liquid-supply pump 324 are configured such that during at least a portion of the negative particle-delivery activation sub-state 348, liquid-supply pump 324 pumps physiological liquid solution 336 at a rate of at least 3 cc/sec, such as at least 7 cc/sec. Further alternatively or additionally, for some applications, control circuitry 332 and mixing pump 322 are configured such that during at least a portion of mixing activation state 342 mixing pump 322 pumps physiological liquid solution 336 at a rate of at least 3 cc/sec, such as at least 7 cc/sec.

For some applications, control circuitry 332 is configured to, before repeatedly assuming mixing and particle-delivery activation states 342 and 344, assume a filling state, in which control circuitry 332 activates liquid-supply pump 324 to apply positive pressure to pump a volume of physiological liquid solution 336 from solid-liquid composition compartment 308 into solid-liquid composition delivery tube 314, the volume equal to between 0.5 and 3 cc.

For some applications, control circuitry 332 is configured to assume mixing activation state 342 and particle-delivery activation state 344 at partially-overlapping times. For some of these applications, control circuitry 332 is configured to assume negative particle-delivery activation sub-state 348 and particle-delivery activation state 344 at partially-overlapping times. For example, control circuitry 332 may be configured to:
begin negative particle-delivery activation sub-state 348 toward the end of mixing activation state 342 (e.g., within the last 30% of mixing activation state 342),
complete negative particle-delivery activation sub-state 348 either simultaneously with the completion of mixing activation state 342, or after the completion of mixing activation state 342, and
begin positive particle-delivery activation sub-state 350 upon the completion of negative particle-delivery activation sub-state 348, typically immediately upon the completion of negative particle-delivery activation sub-state 348.

For some applications, control circuitry 332 is configured to assume mixing activation state 342 and particle-delivery activation slate 344 at the same time.

Reference is made to FIGS. 1C, 2C, and 6C-D. For some applications, control circuitry 332 is configured:

when in positive particle-delivery activation sub-state 350, to activate pump 523 to pump a volume of between 0.1 and 4 cc of fluid (e.g., between 0.1 and 2 cc, such as between 0.2 and 0.9 cc, such as between 0.3 and 0.6 cc), and
when in negative particle-delivery activation sub-state 348, to activate pump 523 to pump the volume of fluid.

Alternatively or additionally, for some applications, control circuitry 332 and pump 523 are configured such that throughout mixing activation state 342 (i.e., during each occurrence of mixing activation state 342 in configurations in which mixing activation state 342 occurs more than once in alternation with particle-delivery activation state 344), pump 523 pumps between 0.5 and 9 cc of physiological liquid solution 336, such as between 1.8 and 3.9 cc of physiological liquid solution 336.

For some applications, liquid-supply pump 324 or the exactly one pump 523 is activated to provide mainly or only positive pressure. In these applications, liquid-supply pump 324 or the exactly one pump 523 may be activated in reverse, to create back flow in solid-liquid composition delivery tube 314 to free blockage of solid-liquid composition delivery tube 314 that may occur because of accumulation of solid bone graft particles 334. Such activation may be periodic, for example as one or few short reverse pulses in each activation sub-state 344. Alternatively, such activation may be periodic, for example as one or few short reverse pulses in some of activation sub-states 344, for example in every other activation sub-state 344 or in one of few activation sub-states 344. Alternatively or additionally, such activation may be activated on demand by the user on viewing accumulation of solid bone graft particles 334 or interruption of flow in the solid-liquid composition delivery tube 314. Alternatively or additionally, control circuitry 332 may activate such activation automatically upon detection of an interruption of flow in solid-liquid composition delivery tube 314 by measuring the pressure in the system, or the resistance at liquid-supply pump 324 or the exactly one pump 523.

Reference is now made to FIGS. 8A-B, which are schematic illustrations of chamber 302, in accordance with an application of the present invention. In this configuration, chamber 302 comprises a receptacle component 370 and a cover component 372. Cover component 372 (a) comprises filter 304, and (b) is shaped so as to define (i) cap 374 and (ii) a bone-graft container 376 having an opening 378 that (x) faces away from cap 374 and (y) is farther from cap 374 than filter 304 is from cap 374. Receptacle component 370 and cover component 372 are shaped so as to be reversibly coupleable with each another to form a watertight seal, with bone-graft container 376 disposed within receptacle component 370.

Before receptacle component 370 and cover component 372 are coupled to each another, bone-graft container 376 contains solid bone graft particles 334. For some applications, such as when bone-graft container 376 is provided pre-loaded with solid bone graft particles 334, bone-graft container 376 further comprises a temporary cap (not shown). For some applications, bone-graft container 376 is placed upside-down on a surface, such that opening 378 is facing up. The temporary cap, if provided, is removed. Receptacle component 370 of chamber 302 is coupled to bone-graft container 376 while bone-graft container 376 remains upside-down. Typically, chamber 302 is turned over to its upright operational position only after bone graft injection system 320 has filled the chamber with physiological liquid solution 336 in the filling state described above.

For some applications, bone-graft container 376 has a volume of between 0.2 and 6 ml. Alternatively or additionally, for some applications, chamber 302 has a volume of between 0.2 and 20 ml. Further alternatively or additionally, for some applications, a volume of bone-graft container 376 equals at least 10% of and/or less than 50% of a volume of chamber 302, such as less than 33%, e.g., less than 20% of the volume of chamber 302.

Reference is now made to FIG. 9, which is a schematic illustration of a portion of a method of using bone graft injection system 320, in accordance with an application of the present invention. All or a portion of these techniques may also be used with bone graft injection systems 420 and 520, mutatis mutandis. A bore 86 (e.g., exactly one bore) is formed through bone 82 from a first side of the bone to a second side of the bone. Schneiderian membrane 88 is raised to form cavity 90 between the second (upper) side of the bone and Schneiderian membrane 88, such as using hydraulic pressure or mechanical elevation, either using shaft unit 340 of bone graft injection system 320 (typically by injecting physiological solution through shaft delivery tube 380 after inserting shaft delivery tube 380 into bore 86), or using another dental tool or a dental implant. For some applications, the surgeon reflects gingiva 84, exposing an occlusal surface of maxillary alveolar bone 82 as shown in FIG. 9. Alternatively, a flapless procedure is performed, in which the gingiva is not reflected (approach not shown). Although a crestal approach is shown, a lateral approach may alternatively be used.

In blow-up A of FIG. 9, Schneiderian membrane 88 has settled toward the bottom of cavity 90, such as after injected saline solution has been allowed to drain from cavity 90 through the tool and/or the bore through the bone.

For some applications, user interface 335 of bone graft injection system 320 includes one or more of the following user controls (which may comprise, for example, buttons), for performing the following functions during use of bone graft injection system 320 in a bone augmentation procedure:
a “load” user control, which instructs control circuitry 332 to fill all of the tubes of bone graft injection system 320 with physiological liquid solution 336, during the filling state described above with reference to FIG. 7;
a “volume” user control, which specifies the maximum volume of physiological liquid solution 336 to be injected into cavity 90 by control circuitry 332;
a “raise” user control, which instructs control circuitry 332 to raise Schneiderian membrane 88 by injecting the volume of physiological liquid solution 336 specified by the “volume” user control (the user activates the “raise” user control when removable depth limiting element 384 is attached to shaft delivery tube 380 and shaft delivery tube 380 is disposed as described hereinbelow with reference to blow-up B of FIG. 9);
a “start” user control, which instructs control circuitry 332 to deliver solid bone graft particles 334 into cavity 90, as described herein (the user activates the “start” user control after removing removable depth limiting element 384 from shaft delivery tube 380 and advancing shaft delivery tube 380 into cavity 90, as described hereinbelow with reference to blow-up C of FIG. 9);
a “stop” user control, which instructs control circuitry 332 to cease delivering solid bone graft particles 334; and
an “empty” user control, which instructs control circuitry 332 to pump all of physiological liquid solution 336 from the system.

For some applications, a method of using bone graft injection system 320 comprises inserting, from a first (lower) side of maxillary bone 82 of a jaw, shaft delivery tube 380 of shaft unit 340 of bone graft injection system 320, 420, or 520 into bore 86 that passes through maxillary bone 82 from the first (lower) side to the second (upper) side of maxillary bone 82, such that distal opening 383 of shaft delivery tube 380 is disposed in bore 86 or in cavity 90 that is (a) adjacent to the second side of maxillary bone 82 and (b) between the second side of maxillary bone 82 and Schneiderian membrane 88. (As mentioned hereinbelow, distal opening 383 is in fluid communication with shaft delivery tube 380.) For some applications, distal opening 383 is disposed at the distal end of shaft delivery tube 380, and positioning distal opening 383 comprises positioning the distal end of shaft delivery tube 380 at the location.

For some applications, a screw 408 that defines a channel is screwed into bore 86 before insertion of shaft delivery tube 380, and shaft delivery tube 380 is inserted into bore 86 by being inserted into the channel of screw 408. Optionally, saline solution was previously injected through the channel of the screw in order to raise Schneiderian membrane 88. For some applications, a seal (e.g., comprising an o-ring) is provided between the wall of the channel and an external surface of shaft delivery tube 380. Alternatively or additionally, a seal is provided against the first (lower) side of first maxillary bone 82. Alternatively, shaft delivery tube 380 is inserted directly through bore 86, without the use of the screw, such as shown in FIGS. 3D-H and FIG. 13.

The method typically further comprises positioning distal opening 383 near a roof 406 of cavity 90. For example, distal opening 383 may be positioned at a solid-liquid-composition-delivery location 410 at a distance D5 from the second side of maxillary bone 82, the distance D5 equal to at least 50% (e.g., at least 75%) of a height H of cavity 90 directly above bore 86. Alternatively or additionally, for some applications, distal opening 383 is positioned at a distance D6 between 2 and 12 mm, such as between 4 and 6 mm from Schneiderian membrane 88 at roof 406 of cavity 90 directly above bore 86. Providing such spacing between distal opening 383 and Schneiderian membrane 88 may prevent solid-liquid composition 339 from rebounding off the membrane directly hack into distal opening 383 before solid bone graft particles 334 can settle in the cavity.

The method further comprises providing solid-liquid composition 339 from a solid-liquid composition source, such as chamber 202 and other elements of bone graft injection system 320 that are coupled in fluid communication with shaft delivery tube 380, typically by activating pump unit 201, such as by activating control circuitry 332. While distal opening 383 is positioned at solid-liquid-composition-delivery location 410, solid-liquid composition 339 is injected through distal opening 383 via shaft delivery tube 380. After solid-liquid composition 339 is injected, an implant is implanted al least partially within cavity 90, either during the same procedure or after bone grows into bone graft particles 334 in cavity 90. After bone grows into bone graft particles 334, a dental appliance, such as a crown, is coupled to the implant.

For some applications, the method further comprises raising Schneiderian membrane 88 by injecting physiological liquid solution 336 through shaft delivery tube 380, such as shown in blow-up B of FIG. 9. For some applications, raising Schneiderian membrane 88 comprises positioning distal opening 383 at a liquid-delivery location 404 that is within bore 86 or within 1 mm above bore 86; and, while distal opening 383 is positioned at liquid-delivery location 404, injecting physiological liquid solution 336 to raise Schneiderian membrane 88. Distal opening 383 is positioned at solid-liquid-composition-delivery location 410 after finishing injecting physiological liquid solution 336 to raise Schneiderian membrane 88.

For some applications, distal opening 383 is positioned at liquid-delivery location 404 while removable depth limiting element 384 is attached to shall delivery tube 380. Removable depth limiting element 384 limits advancement of shaft delivery tube 380 through bore 86. Positioning distal opening 383 at solid-liquid-composition-delivery location 410 comprises removing depth limiting element 384 from shaft delivery tube 380, and subsequently advancing shaft delivery tube 380 through bore 86 until distal opening 383 reaches solid-liquid-composition-delivery location 410, such as shown in blow-up C of FIG. 9.

For some applications, injecting solid-liquid composition 339 comprises pumping solid-liquid composition 339 through distal opening 383 via shaft delivery tube 380 at a pulsating hydraulic pressure that periodically varies between positive and negative. For some applications, solid-liquid composition 339 is injected and drained using techniques described hereinabove with reference to FIGS. 3D-H and/or hereinbelow with reference to FIG. 13.

For some applications of the configuration of FIGS. 1A and 2A, while solid-liquid composition 339 is injected, chamber 302 is oriented such that liquid compartment 306 is above solid-liquid composition compartment 308. Typically, when chamber 302 is oriented such that liquid compartment 306 is above solid-liquid composition compartment 308: (a) the one or more solid-liquid composition ports 312 are disposed no more than a distance from a bottom of solid-liquid composition compartment 308, the distance equal to 25% of a vertical height of solid-liquid composition compartment 308, and/or (b) the one or more solid-liquid composition ports 312 are located through a side wall of solid-liquid composition compartment 308. Typically, while solid-liquid composition 339 is injected, solid-liquid composition delivery tube 314 is oriented within 45 degrees of horizontal, such as within 15 degrees of horizontal, e.g., horizontally. (As used in the present application, including in the claims, “horizontal” means horizontal with respect to the Earth, i.e., perpendicular to a vertical line directed to the center of gravity of the Earth, e.g., as ascertained using a plumb-line.)

Reference is now made to FIG. 10, which is a schematic illustration of a hydraulic sinus lift unit 430, in accordance with an application of the present invention. Hydraulic sinus lift unit 430 is used for applying hydraulic pressure to raise Schneiderian membrane 88 to form cavity 90 between the second (upper) side of the bone and Schneiderian membrane 88.

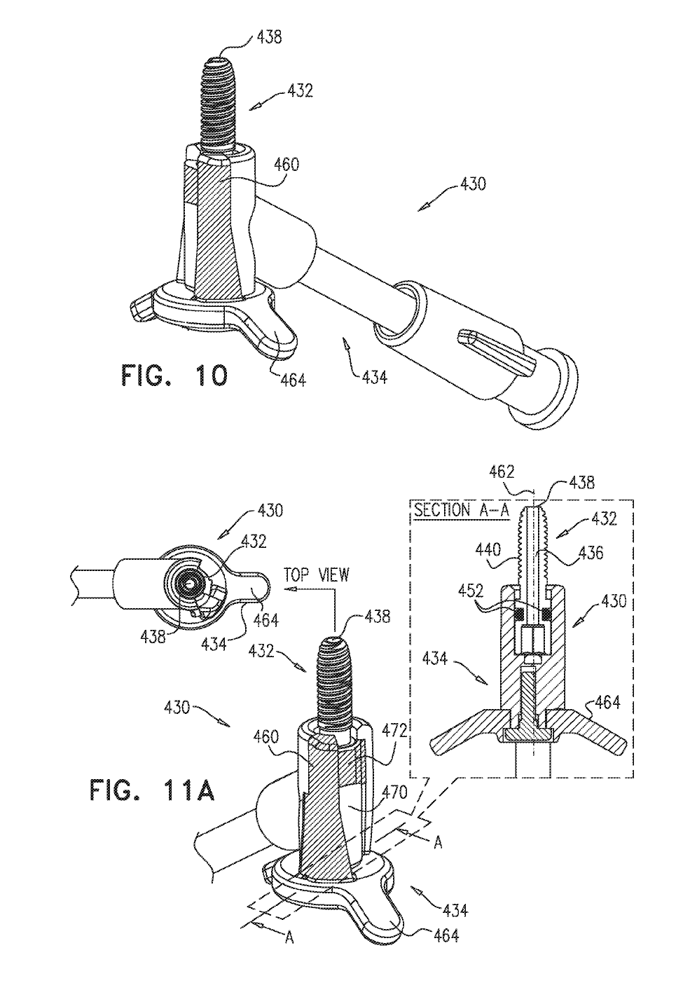

Hydraulic sinus lift unit 430 comprises an osteotome 432 and a fluid-delivery assembly 434, which is removably coupleable to osteotome 432, typically without screwing fluid-delivery assembly 434 onto osteotome 432. Fluid-delivery assembly 434 is shown coupled to osteotome 432 in FIG. 10 (and FIGS. 11A-B and 12B); in FIG. 12A, osteotome 432 is shown along, prior to coupling to fluid-delivery assembly 434.

Reference is also made to FIGS. 11A-B, which are schematic illustrations of hydraulic sinus lift unit 430, with fluid-delivery assembly 434 in unlocked and locked states, respectively, in accordance with an application of the present invention.

Osteotome 432 is shaped so as define a channel 436 therethrough that has a distal opening 438 at or near a distal tip of osteotome 432. Typically, a portion of an external surface of osteotome 432 is shaped so as to define a screw thread 440, for forming a tight liquid seal with the wall of bore 86 through bone 82. Osteotome 432 typically comprises a metal such as stainless steel or titanium, or a ceramic. A proximal end of osteotome 432 defines an internal coupling surface (as shown) and/or an external coupling surface, for coupling with a dental wrench; for example, the coupling surface may be polygonal, e.g., hexagonal.

Fluid-delivery assembly 434 is configured to be coupled to osteotome 432 when fluid-delivery assembly 434 is in an unlocked state, such as shown in FIG. 11A. Fluid-delivery assembly 434 is configured to be transitioned to a locked state, such as shown in FIG. 11B, in which fluid-delivery assembly 434 is tightly locked to osteotome 432, and forms a fluid-tight seal between an internal channel 450 of fluid-delivery assembly 434 and channel 436 of osteotome 432, such as using at least one o-ring 452. Typically, fluid-delivery assembly 434, when in the locked state, can rotate (swivel) about a central longitudinal axis 462 of osteotome 432, which may ease use of operation. A syringe 480 (shown in FIG. 12B) is attached to the distal end of fluid-delivery assembly 434. While the fluid-delivery assembly 434 is in the locked state, the syringe is used to force fluid through osteotome 432 in order to lift the membrane. Typically, locking element 460 allows fast coupling and decoupling of osteotome 432 to and from fluid-delivery assembly 434.

For some applications, fluid-delivery assembly 434 is configured to bidirectionally transition between the unlocked and locked states by rotation of a locking element 460 about central longitudinal axis 462 of osteotome 432. Optionally, fluid-delivery assembly 434 comprises a knob 464 fixed to locking element 460, for rotating locking element 460. For some applications, a body 470 of fluid-delivery assembly 434 is shaped so as to define a sloped portion 472 that (a) pushes locking element 460 away from central longitudinal axis 462 when locking element 460 is rotated at a rotational position corresponding with the unlocked state, such as shown in FIG. 11A, and (b) allows locking element 460 to move toward central longitudinal axis 462 when locking element 460 is rotated at a rotational position corresponding with the locked state, such as shown in FIG. 11B. For some applications, locking element 460 is biased to bend inward toward central longitudinal axis.

Reference is made to FIGS. 12A-B, which are schematic illustrations of a method of using hydraulic sinus lift unit 430 to perform a portion of a sinus lift procedure, in accordance with an application of the present invention. A bore 86 is formed through bone 82, e.g., using techniques described in any of the applications incorporated by reference hereinbelow. As shown in FIG. 12A, before being coupled to fluid-delivery assembly 434, osteotome 432 is screwed into bore 86, typically using a conventional dental wrench. Because fluid-delivery assembly 434 has not yet been coupled to osteotome 432, good access to the osteotome is available for screwing the osteotome into the bore.

As shown in FIG. 12B, fluid-delivery assembly 434 is coupled to osteotome 432 while in the unlocked state, and transitioned to the locked state. During coupling of fluid-delivery assembly 434 to osteotome 432, little or no force, including little or no torque, is applied to osteotome 432, which might dislodge the osteotome or damage the bone, to which the osteotome is already engaged before the coupling process. Fluid-delivery assembly 434 is also coupled to a pressurized fluid source, such as a conventional syringe 480, such that internal channel 450 of fluid-delivery assembly 434 is coupled in fluid communication with the pressurized fluid source. Schneiderian membrane 88 is raised to form cavity 90 between the second (upper) side of the bone and Schneiderian membrane 88, by injecting physiological solution through internal channel 450 of fluid-delivery assembly and channel 436 of osteotome 432.

Reference is still made to FIGS. 10, 11A-B and 12A-B. In an application of the present invention, apparatus is provided that comprises fluid-delivery assembly 434 removably coupleable to osteotome 432 (typically to a distal end of osteotome 432), comprising locking element 460, configured to lock osteotome 432 to fluid-delivery assembly 434 in a locked state and to release osteotome 432 from fluid-delivery assembly 434 in an unlocked state. Channel 436 of osteotome 432 is in sealed fluid communication with fluid-delivery assembly 434 when in the locked state. Fluid-delivery assembly can rotate about a central longitudinal axis of osteotome 432 while in the locked state. Typically, locking element 460 allows fast coupling and decoupling of osteotome 432 to and from fluid-delivery assembly 434.

Reference is made to FIG. 13, which is a schematic illustration of a configuration of and method of using shaft unit 340, in accordance with an application of the present invention. This method may also be used with the configurations of shaft unit 340 described hereinabove with reference to FIGS. 3D-H, which may implement any of the features described with reference to FIG. 13, mutatis mutandis. In this configuration, shaft unit 340 is shaped so as to define a delivery lumen 542 and a drainage lumen 544, as shown in Section B-B and described in more detail hereinbelow. Solid-liquid composition 339 is injected through delivery lumen 542 and distal opening 383 into cavity 90, such that (a) a portion of physiological liquid solution 336 drains into drainage lumen 544, and (b) passage of bone graft particles 334 of solid-liquid composition 339 into drainage lumen 544 is inhibited, such that bone graft particles 334 accumulate in cavity 90, and function as regenerative material. Typically, at least 80% of physiological liquid solution 336 drains in a distal-to-proximal direction, optionally while solid-liquid composition 339 is being injected. Typically, between 0.2 and 20 ml of bone graft particles accumulate in the cavity. Typically, but not necessarily, physiological liquid solution 336 drains into drainage lumen 544 at the same time that solid-liquid composition 339 is injected.

For some applications, exactly one shaft unit 340 is inserted into bore 86 such that rib elements 576 space an external surface 578 of shaft delivery tube 380 away from an inner wall of bore 86, thereby defining a fluid flow path 579 between external surface 578 of shaft delivery tube 380 and the inner wall of bore 86. As a result, (a) the portion of physiological liquid solution 336 drains through fluid flow path 579 and into drainage lumen 544, which typically radially surrounds shaft delivery tube 380, and (b) passage of bone graft particles 334 of solid-liquid composition 339 into fluid flow path 579 is inhibited, such that the solid particles accumulate in the cavity. Typically, an external shaft 554 is disposed radially around shaft delivery tube 380, such that external shaft 554 and shaft delivery tube 380 define drainage lumen 544 radially between external shaft 554 and shaft delivery tube 380. This drainage of excess physiological liquid solution 336 may enable the pumping of more fluid during positive particle-delivery activation sub-state 350 than during negative-positive particle delivery cycles 346, as described hereinabove with reference to FIGS. 4A-B, 5A-D, and 6A-D.

Although the surgical tools and methods described herein have been generally described for sinus lift dental applications, these tools and methods may additionally be used for other dental applications, such as ridge augmentation (in both the maxilla and mandible) (such as by injecting the solid-liquid composition between the gingiva and the bone crest), or sinus floor elevation. In addition, these tools and methods may additionally be used for non-dental applications, such as orthopedic applications. For orthopedic applications, bone graft particles 334 may have a larger average particle size, e.g., up to 7 mm.

Reference is now made to FIG. 14, which is a schematic illustration of one use of bone graft injection system 220 or ridge augmentation, in accordance with an application of the present invention. In this application, bone graft injection system 220, in any of the configurations described herein, is used to perform ridge augmentation of a jaw bone 290 (either a mandible or a maxilla). For some applications, gingiva 292 is dissected from jaw bone 290, such as by tunneling, as is known in the art. Optionally, a structural support 294 is placed under gingiva 292; for example, structural support 294 may comprise a mesh, reinforced membrane, and/or stent. Composition delivery source 200 of bone graft injection system 220 is used to inject solid-liquid composition 339 between jaw bone 290 and gingiva 292, or between jaw bone 290 and structural support 294.

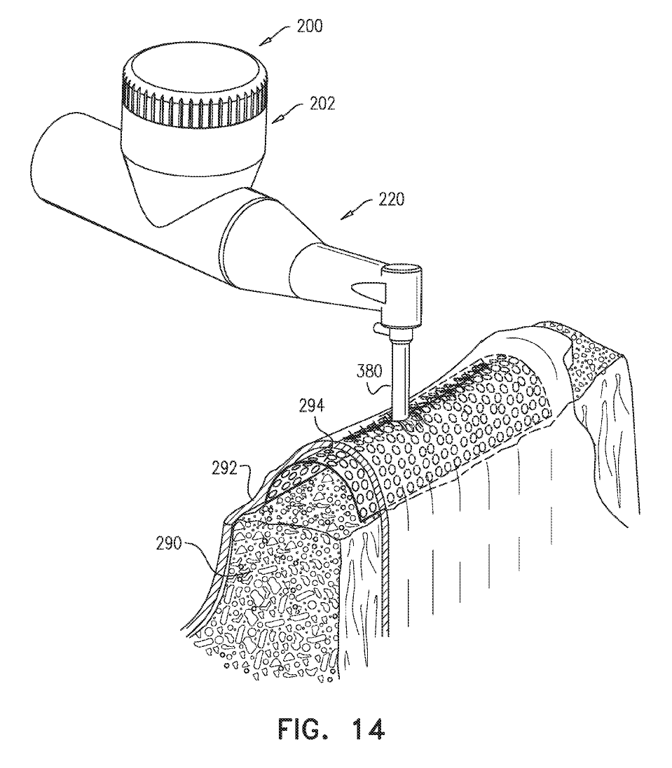

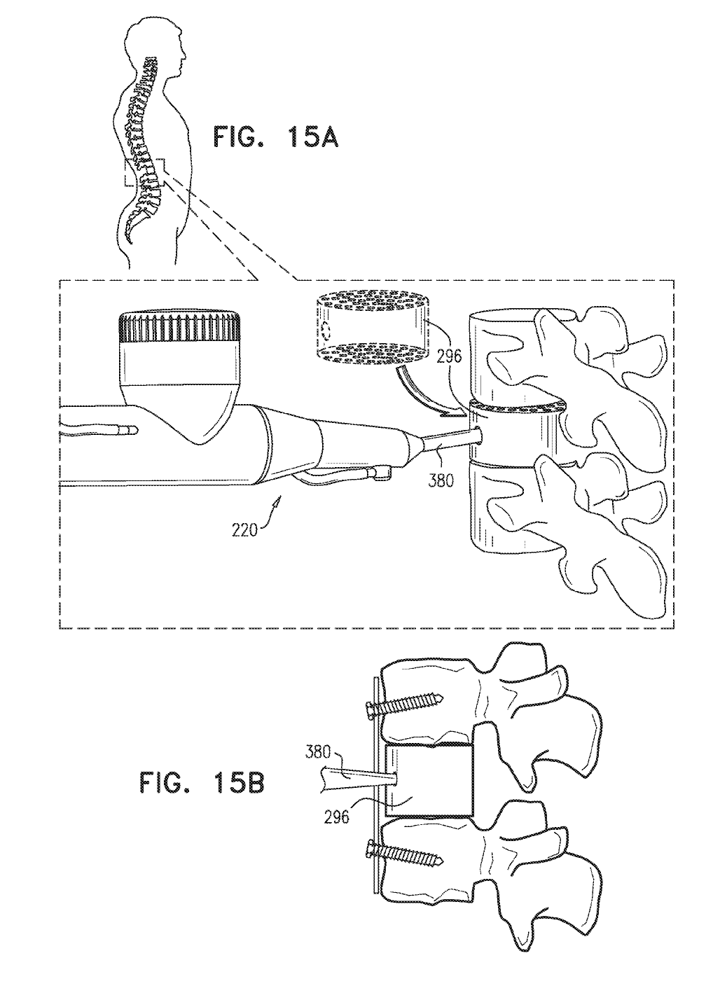

Reference is now made to FIGS. 15A-B, which are schematic illustrations of one use of bone graft injection system 220 for performing a minimally-invasive spinal interbody fusion, in accordance with an application of the present invention. Any of the configurations of bone graft injection system 220 described herein may be used. The approach to the spine (anterior, posterior, or lateral) depends on the site (e.g., lumbar, cervical, or thoracic spine). Typically, an inner vertebral disc is removed or partially removed and replaced with a structural support 296, such as a rigid cage. Composition delivery source 200 of bone graft injection system 220 is used to inject solid-liquid composition 339 into structural support 296. Optionally, external fixation is also performed to fixate the adjacent vertebrae, as is known in the art, such as shown in FIG. 12B. For this application, shaft delivery tube 380 is generally coaxial with the body of composition delivery source 200, i.e., faces forward rather than sideways; shaft delivery tube 380 may also be somewhat longer than in the configurations shown in FIGS. 1A-14.

Reference is now made to FIG. 16, which is a schematic illustration of one use of bone graft injection system 220 for filling a bone defect, in accordance with an application of the present invention. In this application, bone graft injection system 220, in any of the configurations described herein, is used to fill a defect 500 in a bone 510. This technique may be used for orthopedic procedures, as well as for dental procedures. For some applications, a structural element 512, such as a crib, is placed over defect 500 in order to define a volume to be filled. Composition delivery source 200 of bone graft injection system 220 is used to inject solid-liquid composition 339 into the volume defined by structural element 512. As described hereinabove with reference to FIGS. 15A-B, for this application, shaft delivery tube 380 is generally coaxially with the body of composition delivery source 200, and longer than in the configurations shown in FIGS. 1A-14.

Although the techniques described herein have been generally described for use with bone graft particles, these techniques may also be used with other solid particles, such as, as for example, drug-releasing solid particles or solid drug particles, and/or granules.

The scope of the present invention includes embodiments described in the following patents and patent application publications, which are assigned to the assignee of the present application and are incorporated herein by reference. In an embodiment, techniques and apparatus described in one or more of the following patents or patent application publications are combined with techniques and apparatus described herein:
U.S. Pat. No. 7,934,929 to Better et al.
U.S. Pat. No. 8,029,284 to Better et al.
U.S. Pat. No. 8,662,891 to Uchitel et al.
U.S. Pat. No. 8,388,343 to Better et al.
U.S. Pat. No. 8,702,423 to Better et al.
PCT Publication WO 2010/035270 to Better et al.
PCT Publication WO 2010/146573 to Better et al.
PCT Publication WO 2014/199332 to Fostick et al.
U.S. Provisional Application 62/150,969, filed Apr. 22, 2015
U.S. application Ser. No. 14/707,688, filed May 8, 2015, which published as US Patent Application Publication 2016/0310192 to Uchitel et al.
International Application PCT/IL2016/050423, filed Apr. 20, 2016, which published as PCT Publication WO 2016/170540 to Uchitel et al.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with solid particles and a liquid container containing a physiological liquid solution, the apparatus comprising:
(1) a composition delivery source, which comprises:
(a) a chamber, which is shaped so as to define (A) one or more liquid ports in fluid communication with an interior of the chamber, and (B) one or more solid-liquid composition ports in fluid communication with the interior of the chamber;
(b) a solid-liquid composition delivery tube, which is in fluid communication with at least one of the one or more solid-liquid composition ports;
(c) a mixing tube, which is in fluid communication with at least one of the one or more liquid ports and at least one of the one or more solid-liquid composition ports; and
(d) a liquid-supply tube, which is in fluid communication with at least one of the one or more liquid ports; and
(2) a pump unit, which comprises one or more pumps, which are arranged:
(a) to cause flow in the mixing tube during a mixing activation state, and
(b) to cause flow in the liquid-supply tube during a particle-delivery activation state.

2. The apparatus according to claim 1, wherein the solid particles are solid bone graft particles, and wherein the apparatus is for use with the solid bone graft particles.

3. (canceled)

4. The apparatus according to claim 1, wherein the mixing tube (a) merges with the liquid-supply tube at an exit junction, and (b) is in fluid communication with the at least one of the one or more liquid ports via a portion of the liquid-supply tube.

5. The apparatus according to claim 1, wherein the liquid-supply tube (a) merges with the mixing tube at an exit junction, and (b) is in fluid communication with the at least one of the one or more liquid ports via a portion of the mixing tube.

6. The apparatus according to claim 1, wherein the mixing tube (a) merges with the solid-liquid composition delivery tube at a return junction, and (b) is in fluid communication with the at least one of the one or more solid-liquid composition ports via a portion of the solid-liquid composition delivery tube.

7. The apparatus according to claim 1,
wherein the one or more solid-liquid composition ports comprise (a) a solid-liquid composition delivery port and (b) a solid-liquid composition inlet port,
wherein the mixing tube is in fluid communication with the solid-liquid composition inlet port, and
wherein the solid-liquid composition delivery tube is in fluid communication with the solid-liquid composition delivery port.

8. (canceled)

9. The apparatus according to claim 1, wherein a length of the solid-liquid composition delivery tube equals at least 300% of a sum of a length of the mixing tube and a length of the liquid-supply tube.

10. The apparatus according to claim 1, wherein a length of the solid-liquid composition delivery tube is at least 50 cm.

11. (canceled)

12. The apparatus according to claim 1, wherein the one or more pumps are one or more peristaltic pumps, respectively.

13. The apparatus according to claim 1, wherein the one or more pumps comprise:
a mixing pump, which is arranged to cause the flow in the mixing tube during the mixing activation state; and
a liquid-supply pump, which is arranged to cause the flow in the liquid-supply tube during the particle-delivery activation state.

14. The apparatus according to claim 13, wherein the pump unit further comprises control circuitry, which is configured to repeatedly:
(a) assume the mixing activation state, in which the control circuitry activates the mixing pump to mix the solid particles and the physiological liquid solution in the chamber to form a solid-liquid composition, by pumping the physiological liquid solution through the mixing tube and into the chamber, and
(b) assume the particle-delivery activation state, wherein the control circuitry, during at least a portion of the particle-delivery activation state, activates the liquid-supply pump to apply positive pressure to pump the solid-liquid composition from the chamber into the solid-liquid composition delivery tube.

15-21. (canceled)

22. The apparatus according to claim 1, wherein the pump unit comprises exactly one pump, which is arranged to:
(a) cause the flow in the mixing tube during the mixing activation state, and
(b) cause the flow in the liquid-supply tube during the particle-delivery activation state.

23. The apparatus according to claim 1, wherein the one or more pumps comprise a pump that is arranged to:
(a) cause the flow in the mixing tube during the mixing activation state, and
(b) cause the flow in the liquid-supply tube during the particle-delivery activation state.

24. The apparatus according to claim 23, wherein the pump unit further comprises one or more valves, which are arranged to regulate flow in the liquid-supply tube and in the mixing tube.

25. The apparatus according to claim 24, wherein the one or more valves comprise:
a liquid-supply-tube valve, which is arranged to regulate flow in the liquid-supply tube; and
a mixing-tube valve, which is arranged to regulate flow in the mixing tube.

26. (canceled)

27. The apparatus according to claim 23,
wherein the pump unit further comprises:
(a) control circuitry, which is configured to repeatedly:
(i) assume the mixing activation state, in which the control circuitry activates the pump to mix the solid particles and the physiological liquid solution in the chamber to form a solid-liquid composition, by pumping the physiological liquid solution through the mixing tube and into the chamber, and
(ii) assume the particle-delivery activation state, wherein the control circuitry, during at least a portion of the particle-delivery activation state, activates the pump to apply positive pressure to pump the solid-liquid composition from the chamber into the solid-liquid composition delivery tube
(b) a liquid-supply-tube valve, which is arranged to regulate flow in the liquid-supply tube, and
(c) a mixing-tube valve, which is arranged to regulate flow in the mixing tube, and
wherein the control circuitry is configured to:
(a) during the mixing activation state, close the liquid-supply-tube valve and open the mixing-tube valve, and
(b) during the particle-delivery activation state, open the liquid-supply-tube valve and close the mixing-tube valve.

28. (canceled)

29. The apparatus according to claim 1, wherein the pump unit further comprises control circuitry, which is configured to repeatedly:
(a) assume a mixing activation state, in which the control circuitry activates one of the one or more pumps to mix the solid particles and the physiological liquid solution in the chamber to form a solid-liquid composition, by pumping the physiological liquid solution through the mixing tube and into the chamber, and (b) assume a particle-delivery activation state, wherein the control circuitry, during at least a portion of the particle-delivery activation state, activates one of the one or more pumps to apply positive pressure to pump the solid-liquid composition from the chamber into the solid-liquid composition delivery tube.

30-32. (canceled)

33. The apparatus according to claim 29, wherein the control circuitry is configured, during each of one or more negative-positive particle delivery cycles of the particle-delivery activation state, to assume:
a negative particle-delivery activation sub-state, in which the control circuitry activates one of the one or more pumps to apply negative pressure to pump liquid from the solid-liquid composition delivery tube toward the chamber, and
a positive particle-delivery activation sub-state, in which the control circuitry activates the one of the one or more pumps to apply the positive pressure to pump the solid-liquid composition from the chamber into the solid-liquid composition delivery tube, wherein a direction of pumping of the one of the one or more pumps in the positive particle-delivery activation sub-state is opposite a direction of pumping of the one of the one or more pumps in the negative particle-delivery activation sub-state.

34-42. (canceled)

43. The apparatus according to claim 1, wherein the apparatus further comprises a shaft unit, which comprises a shaft delivery tube in fluid communication with a distal end of the solid-liquid composition delivery tube.

44. The apparatus according to claim 70, wherein the shaft unit further comprises a removable depth limiting element, which is configured to limit a depth of insertion of the shaft delivery tube into the bore when the shaft delivery tube is inserted into the bore.

45. The apparatus according to claim 44,
wherein the shaft unit comprises a shaft delivery tube,
wherein the shaft unit further comprises a sealing element disposed around an external surface of the shaft delivery tube, and
wherein the depth limiting element is removable from the shaft unit without removal of the sealing element.

46-63. (canceled)

64. A method for use with solid particles and a liquid container containing a physiological liquid solution, the method comprising:
providing a composition delivery source, which comprises (a) a chamber, which is shaped so as to define (A) one or more liquid ports in fluid communication with an interior of the chamber, and (B) one or more solid-liquid composition ports in fluid communication with the interior of the chamber; (b) a solid-liquid composition delivery tube, which is in fluid communication with at least one of the one or more solid-liquid composition ports; (c) a mixing tube, which is in fluid communication with at least one of the one or more liquid ports and at least one of the one or more solid-liquid composition ports; and (d) a liquid-supply tube, which is in fluid communication with at least one of the one or more liquid ports;
providing a pump unit, which comprises one or more pumps, which are arranged (a) to cause flow in the mixing tube during a mixing activation state, and (b) to cause flow in the liquid-supply tube during a particle-delivery activation state;
inserting, from a first side of a maxillary bone of a jaw, a shaft delivery tube of a shaft unit into a bore that passes through the maxillary bone from the first side to a second side of the maxillary bone, such that a distal opening of the shaft delivery tube is disposed in the bore or in a cavity that is (a) adjacent to the second side of the maxillary bone and (b) between the second side of the maxillary bone and a Schneiderian membrane, wherein the distal opening is in fluid communication with the delivery tube, and the shaft delivery tube is in fluid communication with a distal end of the solid-liquid composition delivery tube; and
activating the pump unit to:
provide a solid-liquid composition of (a) the solid particles and (b) the physiological liquid solution, and
inject the solid-liquid composition through the distal opening via the shaft delivery tube and the solid-liquid composition delivery tube.

65. (canceled)

66. The method according to claim 64, further comprising raising the Schneiderian membrane to form the cavity. 67-69. (canceled)

70. The apparatus according to claim 43,
wherein the solid particles are solid bone graft particles, and wherein the apparatus is for use with the solid bone graft particles, and
wherein the shaft delivery tube is configured to be inserted, from a first side of a maxillary bone of a jaw, into a bore that passes through the maxillary bone from the first side to a second side of the maxillary bone, such that a distal opening of the shaft delivery tube is disposed in the bore or in a cavity that is (a) adjacent to the second side of the maxillary bone and (b) between the second side of the maxillary bone and a Schneiderian membrane.
